(19) United States
(12) Patent Application Publication
(10) Pub. No.: US 2024/0294482 A1
(43) Pub. Date: Sep. 5, 2024

(54) METHOD FOR CATALYTICALLY ACTIVATING CARBON DIOXIDE AS CARBONYLATION REAGENT WITH INORGANIC SULFUR

(71) Applicant: Inner Mongolia University of Technology, Inner Mongolia (CN)

(72) Inventors: Ning ZHU, Inner Mongolia (CN); Rongting HE, Inner Mongolia (CN); Silliu CHENG, Inner Mongolia (CN); Lili YU, Inner Mongolia (CN); Jiakai WU, Inner Mongolia (CN); Guanghui SHI, Inner Mongolia (CN); Yang WANG, Inner Mongolia (CN); Tingxuan FANG, Inner Mongolia (CN); Hailong HONG, Inner Mongolia (CN); Limin HAN, Inner Mongolia (CN)

(21) Appl. No.: 18/258,666

(22) PCT Filed: Dec. 20, 2021
(86) PCT No.: PCT/CN2021/139810

§ 371 Date: Jun. 21, 2023

(30) Foreign Application Priority Data
Dec. 21, 2020 (CN) 202011522973.3

(51) Int. Cl. C07D 277/68; C07D 235/26; C07D 417/06

(52) U.S. Cl. CPC C07D 277/68; C07D 235/26; C07D 417/06

(57) ABSTRACT

Provided is a method for catalytically activating carbon dioxide as a carbonylation reagent with inorganic sulfur. In the method, carbon dioxide can be used to replace a toxic and harmful carbonylation reagent in the presence of H₂S and an alkali for the synthesis of a carbonyl-containing fine chemical product. The method has a relatively high atomic economy and can reduce the generation of by-products.

### TECHNICAL FIELD

The invention relates to the field of organic synthesis, in particular, the invention provides a method for catalytic activating carbon dioxide as a carbonylation reagent by inorganic sulfur.

### BACKGROUND

The development of new green and sustainable organic synthesis methods has attracted more and more attention. Basic components which are pollution-free and recyclable is critical for such methods. Since CO₂ is a non-toxic, abdunant and recyclable, it has been regarded as an ideal carbon resource. CO₂ is not only the waste gas of fossil fuels, but also a cheap, non-toxic, non-flammable and renewable C₁ resource. From the perspective of green chemistry, due to the unique carbonyl structure of CO₂, using CO₂ as a carbonyl reagent in chemical conversion can provide high value added fine chemicals. Such method could be both an effective way to reduce atmospheric CO₂ concentration, and also an important strategy for sustainable energy development. Therefore, it is important to sustainably use CO₂ in the synthesis of high value added chemical products.

Among the various organic transformations of CO₂, the use of CO₂ in carbonylation synthesize to provide heterocyclic structures containing carbonyl groups has attracted increasing attention. CO₂ is used to replace carbon source gases which are toxic and unsafety to users such as CO and phosgene. In recent years, remarkable progress had been made on carbonylation of C—H bonds with CO₂ due to its' high atomicity and economic value. More importantly, since the carbon valance of CO₂ is higher than that of CO, the CO₂ can be idealizedly regarded as a combination of CO and an oxidant (CO₂=CO+[O]), thus being suitable for carbonylation reactions by redox reactions under neutral conditions, and reducing production costs, heavy metal residues and solve safety hazards.

In summary, there is an urgent need in the art to develop a method for preparing carbonyl compounds using CO₂ as a carbon source.

### SUMMARY

The purpose of the present invention is to develop a method for preparing carbonyl compounds using CO₂ as a carbonyl source.

The invention provides a method for preparing carbonyl compounds using CO₂ as a carbonylation reagent, wherein H₂S is used as a catalyst.

The present invention also provides a method for preparing carbonyl compounds using CO₂ as a carbonylation reagent and H₂S involved in the reaction, wherein H₂S is used as both a catalyst to catalyze the carbonylation reaction and as a reactant in the reaction.

The present invention also provides a method for preparing carbonyl compounds using CO₂ as a carbonylation reagent and H₂S involved in the reaction, wherein H₂S is used as both a catalyst to catalyze the carbonylation reaction and as a reductant in the reaction.

The first aspect of the present invention provides a method for preparing carbonyl compounds using carbon dioxide as a carbonylation reagent, wherein the method is performed in the presence of H₂S and optional a base.

In another preferred embodiment, said method comprises the steps (i) or (ii):

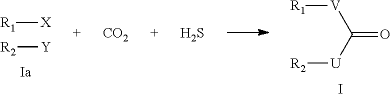

(i) in an optional inert solvent, reacting a compound of formula Ia with CO₂ in the presence of optional a base and an inorganic sulfur reagent to obtain a compound of formula I;

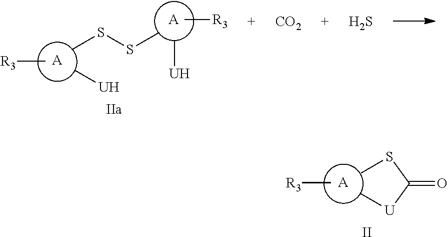

(ii) in an optional inert solvent, reacting a compound of formula IIa with CO₂ in the presence of a base and an inorganic sulfur reagent to obtain a compound of formula II;
wherein, R₁ and R₂ are each independently selected from the group consisting of: substituted or unsubstituted C₁-C₁₂alkyl (e. g. substituted or unsubstituted C₁-C₆alkyl, substituted or unsubstituted C₁-C₈alkyl), substituted or unsubstituted C₃-C₈cycloalkyl, substituted or unsubstituted C₂-C₆alkenyl, and substituted or unsubstituted C₂-C₆alkynyl; or R₁ and R₂ together form a group selected from the group consisting of: substituted or unsubstituted C₁-C₆alkylene, substituted or unsubstituted C₆-C₁₀aryl, and substituted or unsubstituted 5-12-membered heteroaryl;
ring A is substituted or unsubstituted C₆-C₁₀aryl, or substituted or unsubstituted 5-12-membered heteroaryl;
X and Y are independently selected from the group consisting of: halogen, CN, SH, OH, NH₂, NHR, and NO₂;
U and V are independently selected from the group consisting of: NR, S, O, and —C(═S)NH;
R is selected from the group consisting of: H, substituted or unsubstituted C₁-C₁₂ alkyl (such as substituted or unsubstituted C₁-C₆ alkyl, substituted or unsubstituted C₁-C₈ alkyl), substituted or unsubstituted C₃-C₈ cycloalkyl, substituted or unsubstituted C₁-C₆ alkoxy, SO₂CH₃, and phenyl unsubstituted or substituted with 1-4 substituents selected from the group consisting of: halogen, C₁-C₆ alkyl, C₁-C₆ haloalkyl, C₁-C₆ alkoxy, OH, NO₂, NH₂, and SO₂CH₃.

R₃ is one or more groups on the ring A and selected from the group consisting of: H, halogen, C₁-C₆ alkyl, C₁-C₆ alkoxy, C₁-C₆ haloalkyl, NH₂, NO₂, SO₂CH₃, and phenyl unsubstituted or substituted with 1-4 substituents selected from the group consisting of: halogen, C₁-C₆alkyl, C₁-C₆haloalkyl, C₁-C₆alkoxy, OH, NO₂, NH₂, SO₂CH₃; or R₅ and R₆ together form a —(CH₂)ₙ—, wherein, n is selected from 2, 3, 4, 5 or 6;
and the substituted means that one or more hydrogen atoms on the group are substituted with a substituent selected from the group consisting of: halogen, oxygen atom (ie ═O), C₁-C₆alkyl, C₂-C₆alkenyl, C₂-C₆alkynyl, C₁-C₆alkoxy, C₁-C₆haloalkyl, NO₂, SO₂CH₃, phenyl, 5-12-membered heteroaryl, 3-8-membered cycloalkyl, 5-12-membered saturated or partially unsaturated heterocycle; wherein, the phenyl, heteroaryl, cycloalkyl or heterocycle are unsubstituted or substituted by 1-4 substituents selected from the group consisting of: halogen, C₁-C₆alkyl, C₁-C₆haloalkyl, C₁-C₆alkoxy, OH, NO₂, NH₂, and SO₂CH₃;
or, two substituents adjacent or attached to the same carbon atom together form a —(CH₂)ₙ—, wherein, n is selected from 2, 3, 4, 5 or 6.

In another preferred embodiment, the base is an organic base; preferably, the base is selected from the group consisting of: C₁-C₁₂tertiary amines, C₁-C₁₂secondary amines, C₁-C₁₂primary amines, C₂-C₁₂amidines, C₂-C₁₂ guanidines, C₃-C₁₂ pyridines, C₃-C₁₂ imidazoles; preferably, the base is selected from the group consisting of: DBU, TBD, MTBD, DBN, TMG, DABCO, ethylenediamine, triethylamine, DIPEA, DMAP, pyridine, and combinations thereof; preferably, the molar ratio of the reaction substrate to the base is 1:0-5 (e. g., 1:0.1-5).

In another preferred embodiment, the method comprises steps (a), (b), (c), (d), (e), (f) or (g):

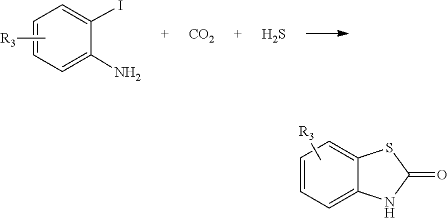

(a) in an optional inert solvent, reacting an o-iodoaniline with CO₂ and H₂S in the presence of a base to obtain a benzothiazolone derivative;

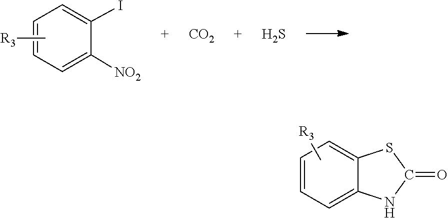

(b) in an optional inert solvent, reacting an o-nitroiodobenzene with CO₂ and H₂S in the presence of a base to synthesize a benzothiazolone derivative;

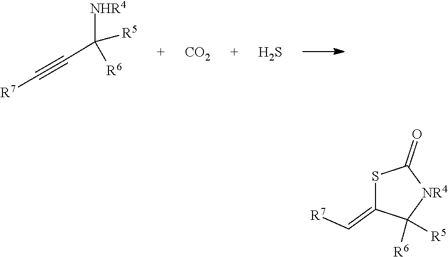

(c) in an optional inert solvent, reacting a propargylamine derivative with CO₂ and H₂S in the presence of an optional base to synthesize a thiazolidin-2-one derivative;
wherein, R₄ is selected from the group consisting of: H, substituted or unsubstituted C₁-C₁₂alkyl, substituted or unsubstituted C₃-C₈cycloalkyl, substituted or unsubstituted phenyl;
R₅, R₆ and R₇ are independently selected from the group consisting of: H, substituted or unsubstituted C₁-C₁₂alkyl, substituted or unsubstituted C₃-C₈cycloalkyl, phenyl, 5-12 membered heteroaryl, and 5-12 membered saturated or partially unsaturated heterocycle, and the phenyl, heteroaryl or heterocycle is unsubstituted or substituted with 1-4 substituents selected from the group consisting of halogen, C₁-C₆alkyl, C₁-C₆haloalkyl, C₁-C₆alkoxy, OH, NO₂, NH₂, SO₂CH₃; or R₅ and R₆ together form a —(CH₂)ₙ—, wherein, n is selected from 2, 3, 4, 5 or 6;

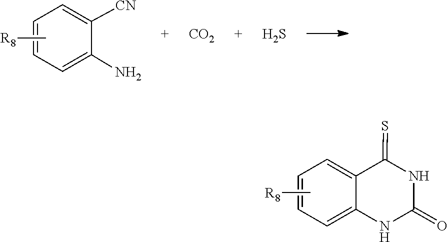

(d) in an optional inert solvent, reacting an o-aminobenzonitrile with CO₂ and H₂S in the presence of a base to synthesize a thioquinazolindione derivative;
wherein, R₈ is one or more substituents on the benzene ring and selected from the group consisting of: H, halogen, C₁-C₆alkyl, C₁-C₆alkoxy, C₁-C₆haloalkyl, NO₂, SO₂CH₃, and phenyl unsubstituted or substituted with 1-4 substituents selected from the group consisting of: halogen, C₁-C₆alkyl, C₁-C₆haloalkyl, C₁-C₆alkoxy, OH, NO₂, NH₂, and SO₂CH₃;

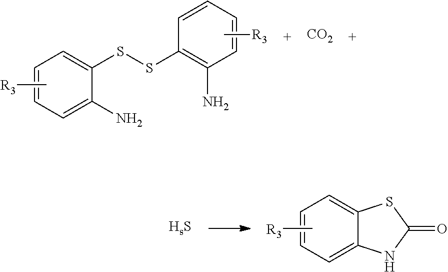

(e) in an optional inert solvent, in the presence of a base, reacting an aromatic o-aminodisulfide with CO₂ in the presence of H₂S to synthesize a benzothiazolone derivative;

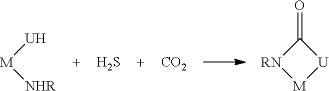

(f) In an optional inert solvent, in the presence of an optional base, reacting an diamine, an alcoholamine or a mercaptoamine with CO₂ in the presence of H₂S to synthesize an imidazolidinone derivative, an oxazolidinone derivative or a thiazolidinone derivative; wherein U is O, S or NR;
M is substituted or unsubstituted C₂-C₄alkylene, substituted or unsubstituted phenyl, or substituted or unsubstituted 5-12 membered heteroaryl, wherein the definition of the substituted is as described in claim 2;
(g) in an optional inert solvent, in the presence of an optional base, reacting an amine with CO₂ in the presence of H₂S to synthesize a urea derivative;

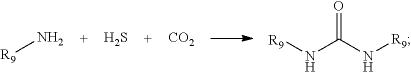

each R₉ are selected from the group consisting of: H, substituted or unsubstituted C₁-C₁₂ alkyl (such as substituted or unsubstituted C₁-C₆ alkyl, substituted or unsubstituted C₁-C₈ alkyl), substituted or unsubstituted C₃-C₈cycloalkyl, phenyl, 5-12-membered heteroaryl, and 5-12-membered saturated or partially unsaturated heterocycle, and the phenyl, heteroaryl or heterocycle is unsubstituted or substituted with 1-4 substituents selected from the group consisting of: halogen, C₁-C₆alkyl, C₁-C₆haloalkyl, C₁-C₆alkoxy, OH, NO₂, NH₂, and SO₂CH₃.

In another preferred embodiment, the inert solvent is selected from the group consisting of: NMP, DMF, THF, DMSO, 1,4-dioxane, HMPA, CH₂Cl₂, CHCl₃, CCl₄, toluene, ethyl acetate, supercritical CO₂, and combinations thereof.

In another preferred embodiment, in the reaction, the molar ratio of the reaction substrate to the CO₂ is 1:1-100.

In another preferred embodiment, during the reaction, the CO₂ is continuously introduced into the reactor, and the pressure of the CO₂ in the reactor is 0.1-12 MPa.

In another preferred embodiment, in the reaction, the molar ratio of the reaction substrate to the H₂S is 1:0.05-20.

In another preferred embodiment, during the reaction, the H₂S is continuously introduced into the reactor, and the pressure of the H₂S in the reactor is 0.05-1.5 MPa.

In another preferred embodiment, in the reaction, the reaction temperature is from room temperature to 150° C.

It should be understood that within the scope of the present invention, the above-described technical features of the present invention and the technical features described in detail below (e.g., embodiments) may be combined with each other to constitute a new or preferred technical solution. Limited to space, I will not repeat them here.

### DETAILED DESCRIPTION OF THE INVENTION

After a long and thorough research, the present inventors unexpectedly found that H₂S can be used as a catalyst to catalyze the reaction of CO₂ as a carbonyl source with a series of substrates to prepare carbonylated compounds with high efficiency. This carbonylation reaction can occur alone or together with other reactions involving CO₂ or H₂S to prepare a series of products, which has potential application value in the field of fine chemical synthesis. Based on the above findings, the inventors completed the present invention.

### Synthesis of CO₂ as Carbonylation Reagents

The present invention provides a method for preparing carbonyl compounds using carbon dioxide as a carbonylation reagent, wherein the method is performed in the presence of H₂S.H₂S can be used as a catalyst only, or as a reactant at the same time as the catalyst, thereby reacting further with the reaction substrate or generating an intermediate.

Specifically, the method comprises step (i) or step (ii):

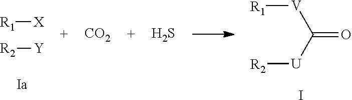

(i) in an inert solvent, reacting a compound of formula Ia with CO₂ in the presence of a base and an inorganic sulfur reagent to obtain a compound of formula I (wherein the compound of formula Ia can be a mixture of R₁—X and R₂—Y, or a compound with two reactive functional groups X and Y formed by R₁—X and R₂—Y together);

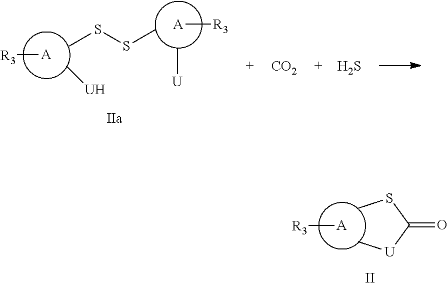

(ii) in an inert solvent, reacting a compound of formula IIa with CO₂ in the presence of a base and an inorganic sulfur reagent to obtain a compound of formula II.
wherein, the definition of each group is as described above.

In a preferred embodiment of the present invention, the step is carried out in the presence of a base, which may preferably be an organic base. Preferably, the base is selected from the group consisting of: C₁-C₁₂ tertiary amines, C₁-C₁₂ secondary amines, C₁-C₁₂ primary amines, C₂-C₁₂ amidines, C₂-C₁₂ guanidines, C₃-C₁₂ pyridines, C₃-C₁₂ imidazoles, DBU, TBD, MTBD, DBN, TMG, DABCO, ethylenediamine, triethylamine, DIPEA, DMAP, pyridine, and combinations thereof; preferably, the molar ratio of the reaction substrate to the base is 1:0.1-5.

In the method, a conventional inert solvent that does not affect the reaction may be used, and the preferred solvent includes: NMP, DMF, THF, DMSO, 1,4-dioxane, HMPA, CH₂Cl₂, CHCl₃, CCl₄, toluene, ethyl acetate, or a combination thereof. In particular, since the method of the invention requires the use of a CO₂ flow, a preferred embodiment is to use supercritical CO₂ as a solvent.

The molar ratio of reaction substrate to CO₂ in the method is not particularly limited, which can be 1:1-100.

In the reaction process, CO₂ is continuously introduced into the reactor. In a preferred reaction method, the pressure of the CO₂ in the reactor is 0.1-12 MPa; such as 0.2-10 MPa, 0.5-10 MPa, 0.6-10 MPa, 0.8-8 MPa, or 1 MPa, 2 MPa, 3 MPa, 4 MPa, 5 MPa or 6 MPa.

In the reaction, the molar ratio of the reaction substrate to the inorganic sulfur is preferably 1:0.05-20.

In the reaction process, H₂S is continuously introduced into the reactor, and preferably the pressure of the H₂S in the reactor is 0.08-1.5 MPa, such as 0.1 MPa, 0.2 MPa, 0.3 MPa, 0.4 MPa, 0.5 MPa, 0.6 MPa, 0.7 MPa, 0.8 MPa, 0.9 MPa, 1 MPa, 1.1 MPa, 1.2 MPa, 1.3 MPa, or 1.4 MPa.

The temperature of the reaction is not particularly limited, and preferably can be carried out at room temperature (typically 0-40° C.) to 150° C., such as 20° C., 30° C., 40° C., 50° C., 60° C., 70° C., 80° C., 90° C., 100° C., 110° C., 120° C., 130° C., or 140° C.

The above reaction can be used to prepare a series of compounds with characteristic structures, for example, compounds with corresponding structural units can be prepared by steps (a), (b), (c), (d), (e) or (f);

### (a) Synthesis of Benzothiazolone Derivatives by the Reaction of o-Iodoaniline with CO₂ and H₂S

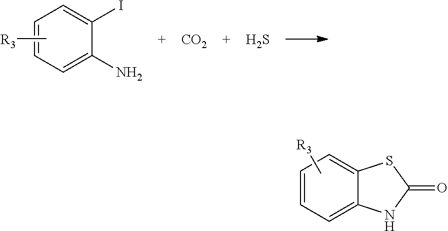

In an inert solvent, reacting an o-iodoaniline with CO₂ and H₂S in the presence of a base to obtain a benzothiazolone derivative. In the above reaction, the base is preferably DABCO, DBU, TBD, or Et₃N; more preferably DBU or Et₃N. The solvent is preferably NMP or DMF, and the amount of base is preferably 1-3 equivalents. In particular, when the reactant is an organic base or can be used as a solvent, the reaction may also be carried out without a solvent or without an organic base.

In another preferred embodiment, in step (a), the pressure ratio of the CO₂ to the H₂S is (1-8):(0.1-0.8).

In another preferred embodiment, in step (a), the process is reacted at 70-100° C., preferably 80-90° C.

In another preferred embodiment, in step (a), in the process, the pressure of the CO₂ in the reactor is 2-5 MPa, and the pressure of the H₂S in the reactor is 0.3-0.5 MPa.

### (b) Synthesis of Benzothiazolone Derivatives by the Reaction of o-Nitroiodobenzene with CO₂ and H₂S

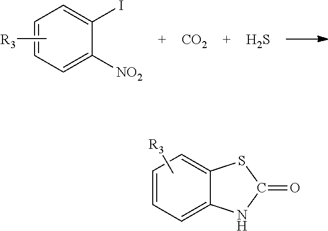

In an inert solvent, reacting an o-nitroiodobenzene with CO₂ and H₂S in the presence of a base to synthesize a benzothiazolone derivative. In the above reaction, the base is preferably DBU or Et₃N; the solvent is preferably NMP or NMP/H₂O; and the amount of base is preferably 2-5 equivalents, preferably 2-4 equivalents.

In another preferred embodiment, in step (b), the pressure ratio of CO₂ to H₂S is (1-8):(0.5-1.5), preferably 2-4:1.

In another preferred embodiment, the reaction may be carried out under CuI catalysis.

In another preferred embodiment, in step (b), the process is reacted at 70-100° C., preferably 80-90° C. In another preferred embodiment, in step (a), in the process, the pressure of the CO₂ in the reactor is 2-5 MPa, and the pressure of the H₂S in the reactor is 0.5-1 MPa.

### (c) Synthesis of Thiazolidin-2-One Derivatives by the Reaction of Propargylamine with CO₂ and H₂S

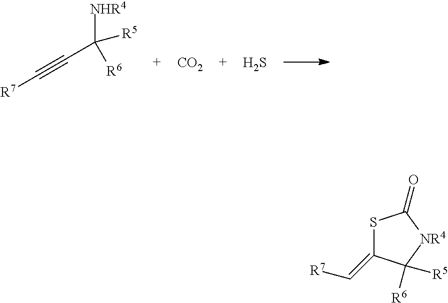

In an inert solvent, reacting an propargylamine derivative with CO₂ and H₂S in the presence of a base to synthesize a thiazolidin-2-one derivative.
wherein, R₄ is selected from the group consisting of: H, substituted or unsubstituted C₁-C₆alkyl;
R₅, R₆ and R₇ are independently selected from the group consisting of: H, substituted or unsubstituted C₁-C₆alkyl, and phenyl unsubstituted or substituted with 1-3 substituents selected from the group consisting of: halogen, C₁-C₆alkyl, C₁-C₆haloalkyl, C₁-C₆alkoxy, OH, NO₂, NH₂, SO₂CH₃; or R₅ and R₆ together form a —(CH₂)ₙ—, wherein, n is selected from 2, 3, 4, 5 or 6;

In the above reaction, the base is preferably DBU, Et₃N, TBD or K₂CO₃, more preferably DBU, Et₃N or TBD; the solvent is preferably CH₃OH, DMF, NMP or DMSO, preferably DMSO; and the amount of the base is preferably 0.5-1.5 equivalents, preferably 0.6-1.2 equivalents.

In another preferred embodiment, in step (c), the pressure ratio of the CO₂ to the H₂S is 1:(0.2-1.5), preferably 1:0.8-1.2.

In another preferred embodiment, in step (c), the process is reacted at 20-60° C., preferably at 20-40° C.

In another preferred embodiment, in step (c), in the process, the pressure of the CO₂ in the reactor is 0.8-1.2 MPa, and the pressure of the H₂S in the reactor is 0.5-1 MPa (preferably 0.8-1 MPa).

### (d) Synthesis of Thiobenzamide or Thioquinazolindione Derivatives by the Reaction of Orthoaminobenzonitrile with CO₂ and H₂S

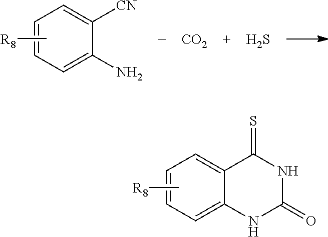

In an inert solvent, reacting an o-aminobenzonitrile with CO₂ and H₂S in the presence of a base to synthesize a thiobenzamide or a thioquinazolindione derivative; in the reaction, CO₂ and H₂S are both used as reactants to form a six-membered ring structure. In the above reaction, the base is preferably DBU; the solvent is preferably DMF, and the amount of the base is preferably 0.2-2 equivalents, preferably 0.8-2 equivalents.

In another preferred embodiment, in step (d), the pressure ratio of CO₂ to H₂S is (2-5):(0.2-1.2), preferably 3-10:1.

In another preferred embodiment, in step (d), the process is reacted at 40-60° C., preferably 45-55° C. In another preferred embodiment, in step (d), in the process, the pressure of the CO₂ in the reactor is 2-5 MPa, and the pressure of the H₂S in the reactor is 0.4-1 MPa.

### (e) Synthesis of Benzothiazolone Derivatives by the Reaction of Aromatic o-Aminodisulfides with CO₂ in the Presence of H₂S

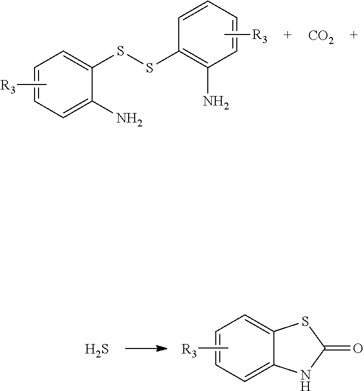

In an inert solvent, in the presence of a base, reacting an aromatic o-aminodisulfide with CO₂ in the presence of H₂S to synthesize a benzothiazolone derivative; in the above reaction, the base is preferably DBU, TMG, or Et₃N; the solvent is preferably NMP, CH₃OH, 1,4-dioxane, or DMSO, more preferably NMP; the amount of base used is preferably 0.2-2 equivalents, more preferably 0.4-1.2 equivalent.

In another preferred embodiment, in step (e), the pressure ratio of the CO₂ to the H₂S is (1-5):(0.1-1.2).

In another preferred embodiment, in step (e), the process is reacted at 25-100° C., preferably at 80-90° C.

In another preferred embodiment, in step (e), in the process, the pressure of the CO₂ in the reactor is 1-5 MPa, and the pressure of the H₂S in the reactor is 0.2-1.0 MPa.

### (f) Synthesis of Imidazolidinone (Oxazolidinone or Thiazolidinone) Derivatives by the Reaction of Diamine, Alcoholamine or Mercaptoamine with CO₂ in the Presence of H₂S

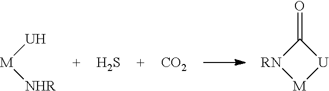 [0084] in an optional inert solvent, in the presence of an optional base, reacting a diamine, an alcoholamine or a mercaptoamine with CO₂ in the presence of H₂S to synthesis an imidazolidinone derivative, an oxazolidinone derivative or a thiazolidinone derivative;
wherein, U is O, S or NR;
M is substituted or unsubstituted C₂-C₄alkylene, substituted or unsubstituted phenyl, or substituted or unsubstituted 5-12 membered heteroaryl, wherein the definition of substitutions are as described above.

### When U is NR and M is a Substituted or Unsubstituted Phenyl, or a Substituted or Unsubstituted 5-12-Membered Heteroaryl Group

In the above reaction, the base is preferably DABCO, DBU, TMG or Et₃N, preferably DBU or TMG; the solvent is preferably NMP, DMF, ethylene glycol or dichloromethane, preferably NMP; and the amount of the base is preferably 0.1-2 equivalents. In particular, when the reactant is an organic base or can be used as a solvent, the reaction may also be carried out without a solvent or without an organic base.

In another preferred embodiment, in step (f), the pressure ratio of CO₂ to H₂S is (1-65):1.

In another preferred embodiment, in step (f), the process is reacted at 20-60° C., preferably at 30-50° C. In another preferred embodiment, in step (f), in the process, the pressure of the CO₂ in the reactor is 1-5 MPa, and the pressure of the H₂S in the reactor is 0.05-1.5 MPa.

### When U is NR and M is a Substituted or Unsubstituted C₂-C₄Alkylene

In the above reaction, the base is preferably DBU, TBD, DIPEA or Et₃N, preferably DBU, DIPEA or Et₃N; the solvent is preferably NMP, DMF, ethylene glycol or dichloromethane, preferably NMP; and the amount of base is preferably 0.1-1 equivalent, more preferably 0.2-0.6 equivalent. In particular, when the reactant is an organic base or can be used as a solvent, the reaction may also be carried out without a solvent or without an organic base.

In another preferred embodiment, in step (f), the pressure ratio of CO₂ to H₂S is (3-35):1, preferably (3-25):1.

In another preferred embodiment, in step (f), the process is reacted at 80-120° C.

In another preferred embodiment, in step (f), in the process, the pressure of the CO₂ in the reactor is 1-5 MPa, and the pressure of the H₂S in the reactor is 0.2-1 MPa.

### When U is S

In the above reaction, the base is preferably DABCO, DBU, TMG, TBD or Et₃N, preferably DBU, Et₃N, or TMG; the solvent is preferably NMP, DMF, ethylene glycol or dichloromethane, preferably NMP or DMF; and the amount of base is preferably 0.1-2 equivalents.

In particular, when the reactant is an organic base or can be used as a solvent, the reaction may also be carried out without a solvent or without an organic base. In addition, it can also be performed in the absence of solvent when the pressure of CO₂ and the reaction temperature are compatible with the conditions for the formation of supercritical CO₂.

In another preferred embodiment, in step (f), the pressure ratio of CO₂ to H₂S is (3-25):1.

In another preferred embodiment, in step (f), the process is reacted at 20-60° C., preferably at 30-50° C.

In another preferred embodiment, in step (f), in the process, the pressure of the CO₂ in the reactor is 3-12 MPa, and the pressure of the H₂S in the reactor is 0.2-1.0 MPa.

### (g) Synthesis of Urea Derivatives by the Reaction of Benzylamine and CO₂ in the Presence of H₂S

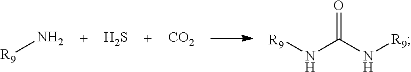 [0100] in an optional inert solvent, in the presence of an optional base, reacting an amine with CO₂ in the presence of H₂S to synthesis an urea derivative.

R₉ are selected from the group consisting of: H, substituted or unsubstituted C₁-C₁₂alkyl (such as substituted or unsubstituted C₁-C₆alkyl, substituted or unsubstituted C₁-C₈alkyl), substituted or unsubstituted C₃-C₈cycloalkyl, phenyl, 5-12-membered heteroaryl, and 5-12-membered saturated or partially unsaturated heterocycle, and the phenyl, heteroaryl or heterocycle is unsubstituted or substituted with 1-4 substituents selected from the group consisting of: halogen, C₁-C₆alkyl, C₁-C₆haloalkyl, C₁-C₆alkoxy, OH, NO₂, NH₂, and SO₂CH₃;

In the above reaction, the base is preferably DBU, TMG or Et₃N, preferably DBU; the solvent is preferably NMP, DMF or methanol, preferably NMP; the amount of base is preferably 0.1-2 equivalents, however, since the substrate organic amine can be used as a base or solvent, the reaction can also be carried out in the absence of an organic base, and/or in the absence of a solvent.

In another preferred embodiment, in step (f), the pressure ratio of CO₂ to H₂S is (5-20):1.

In another preferred embodiment, in step (f), the method is reacted at 90-130° C., preferably at 30-50° C.

In another preferred embodiment, in step (f), in the process, the pressure of the CO₂ in the reactor is 5-20 MPa, and the pressure of the H₂S in the reactor is 0.5-2 MPa.

The present invention is further described below in conjunction with specific embodiments. It should be understood that these embodiments are only used to illustrate the present invention and not to limit the scope of the present invention. The following embodiments do not indicate the specific conditions of the experimental method, usually according to the conventional conditions, or according to the conditions recommended by the manufacturer. Percentages and servings are calculated by weight unless otherwise stated.

### Class I Inorganic Sulfur as Both Raw Material and Catalyst

### Example 1 Synthesis of Benzothiazolone Derivatives by the Reaction of o-Iodoaniline with CO₂ and H₂S

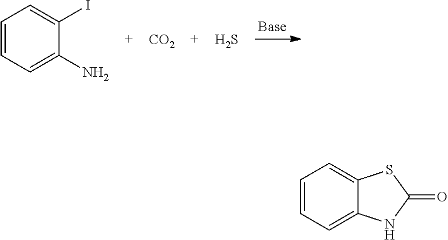

### The Reaction Method is as Follows

1 mol of o-halogenated aniline, 2 mol of base, 0.2 mol of cuprous iodide (CuI) and 2 ml of solvent were weighed and added into the reactor sequentially and the reactor was tightened. The corresponding amount of H₂S was introduced into the reactor, and the reaction was stirred at 90° C. for 30 min, then the corresponding amount of CO₂ was introduced into the reactor, and the reaction was continued to be stirred at the corresponding temperature for 24 h. After the reaction, the reactor was cooled to room temperature, and the reactor was opened after the gas in the reactor was slowly exhausted, then the reaction mixture was transferred to a 250 ml partition funnel and extracted with ethyl acetate. The organic phase was dried with anhydrous magnesium sulfate, then separated by column chromatography to obtained the product.

The conditions were optimized according to the above steps, and the reaction results are shown in the following table:

| | Sol- | | T | molar ratio (o- | P(MPa) | | Yield |
|---|---|---|---|---|---|---|---|
| Entry | vent | base | (° C.) | iodoaniline:DBU) | CO₂ | H₂S | (%) |
| 1 | NMP | DABCO | 90 | 1:2 | 3 | 0.3 | 0 |
| 2 | NMP | DBU | 90 | 1:2 | 3 | 0.3 | 95 |
| 3 | NMP | TBD | 90 | 1:2 | 3 | 0.3 | 56 |
| 4 | NMP | Et₃N | 90 | 1:2 | 3 | 0.3 | 90 |
| 6 | NMP | — | 90 | 1:0 | 3 | 0.3 | 0 |
| 7 | NMP | DBU | 90 | 1:2 | 5 | 0.3 | 87 |
| 8 | NMP | DBU | 90 | 1:2 | 2 | 0.3 | 88 |
| 9 | NMP | DBU | 90 | 1:2 | 3 | 0 | 0 |
| 10 | NMP | DBU | 90 | 1:2 | 3 | 0.5 | 90 |
| 11 | DMF | DBU | 90 | 1:2 | 3 | 0.5 | 76 |
| 12 | NMP | DBU | 80 | 1:2 | 3 | 0.5 | 89 |
| 13 | NMP | DBU | 70 | 1:2 | 3 | 0.5 | 78 |
| 14 | NMP | DBU | 100 | 1:2 | 3 | 0.5 | 91 |

Note:
In each of the above entries, the raw material was 1 mmol o-iodoaniline; the solvent used was 2 ml; CuI used was 0.2 mmol; and reacted for 24 h.

### Using the Method as in Entry 2 Above While Changing Other Reaction Substrates, the Following Individual Products were Obtained

### Characterization of Compounds

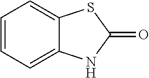

Separated by dry packing and dry sample loading column chromatography (200-300 mesh silica gel): petroleum ether:ethyl acetate (V/V)=4:1 as developing solvent, 144 mg of white solid benzothiazolone was obtained with a yield after separation of 95%.

Characterization data of benzothiazol-2-one (2a): ¹H NMR (CDCl₃, 500 MHz): δ (ppm) 10.01 (brs, 1H), 7.41 (d, 1H, J=7.5 Hz), 7.30-7.26 (m, 1H), 7.17-7.14 (m, 2H). ¹³C NMR (CDCl₃, 125 MHz): δ (ppm) 172.8, 135.3, 126.5, 123.9, 123.3, 122.6, 111.7; MS (EI): m/z calcd for C₇H₅NOS [M]⁺: 151.0, found 151.0. m.p.: 139-140° C.

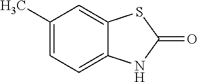

Separated by dry packing and dry sample loading column chromatography (200-300 mesh silica gel): petroleum ether:ethyl acetate (V/V)=3:1 as developing agent, 138 mg of white solid was obtained with a yield after separation of 84%.

Characterization data of 6-methylbenzothiazol-2-one:¹H NMR (DMSO-d₆, 500 MHz): δ (ppm) 11.75 (brs, 1H), 7.36 (s, 1H), 7.07-7.09 (m, 1H), 7.00(d, 1H, J=8 Hz), 2.30 (s, 3H); ¹³C NMR (DMSO-d₆, 125 MHz): δ (ppm) 169.8, 133.9, 131.7, 127.0, 123.2, 122.5, 111.1, 20.5; MS (ESI): m/z calcd for C8H7NOS [M+1]⁺: 166.0, found 165.0. m.p.: 170-171° C.

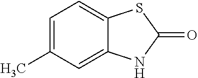

Separated by dry packing and dry sample loading column chromatography (200-300 mesh silica gel): petroleum ether:ethyl acetate (V/V)=2:1 as developing agent, 13 4 mg of white solid was obtained with a yield after separation of 82%.

Characterization data of 5-methylbenzothiazole-2-one:¹H NMR (DMSO-d₆, 500 MHz): δ (ppm) 11.79 (s, 1H), 7.42 (d, J=7.9 Hz, 1H), 6.98-6.91 (m, 2H), 2.32 (s, 3H). ¹³C NMR (DMSO-d₆, 126 MHz) δ (ppm) 170.31, 136.34, 136.04, 123.49, 122.37, 119.95, 111.84, 20.98; MS (ESI): m/z calcd for C8H7NOS [M+1]+: 166.1, found 165.0.

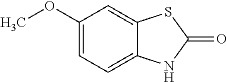

Separated by dry packing and dry sample loading column chromatography (200-300 mesh silica gel): petroleum ether:ethyl acetate (V/V)=3:1 as developing agent, 158 mg of white solid was obtained with a yield after separation of 88%.

Characterization data of 6-methoxybenzothiazol-2-one:¹H NMR (DMSO-d₆, 500 MHz): δ (ppm) 11.66 (brs, 1H), 7.23 (d, 1H, J=2.5 Hz), 7.02 (d, 1H, J=8.5 Hz), 6.86 (dd, 1H, J₁=8.5 Hz, J₂=2.5 Hz), 3.73(s, 3H); 13C NMR (DMSO-d₆, 126 MHz): δ (ppm) 169.8, 155.2, 129.9, 124.3, 113.2, 112.1, 107.8, 55.6; MS (ESI): m/z calcd for C8H7NO2S [M+1]⁺: 182.1, found 181.1.

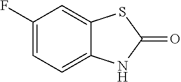 [SMILES: O=c1[nH]c2ccc(F)cc2s1]

Separated by dry packing and dry sample loading column chromatography (200-300 mesh silica gel): petroleum ether:ethyl acetate (V/V)=3:1 as developing agent, 144 mg of white solid was obtained with a yield after separation of 85%.

Characterization data of 6-fluorobenzothiazole-2-one: ¹H NMR (500 MHz, DMSO-d₆) δ (ppm) 11.91 (s, 1H), 7.57 (ddt, J=9.1, 2.2, 0.8 Hz, 1H), 7.16-7.08 (m, 2H). ¹³C NMR (DMSO-d₆, 126 MHz) δ (ppm) 169.83, 157.89 (d, J=119.3 Hz), 132.85 (d, J=1.9 Hz), 124.66 (d, J=5.5 Hz), 113.55 (d, J=12.0 Hz), 112.38 (d, J=4.3 Hz), 109.95 (d, J=13.7 Hz). MS (ESI): m/z calcd for C7HFNOS [M+1]+: 170.1, found 169.1.

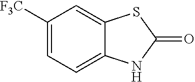 [SMILES: O=c1[nH]c2ccc(C(F)(F)F)cc2s1]

Separated by dry packing and dry sample loading column chromatography (200-300 mesh silica gel): petroleum ether:ethyl acetate (V/V)=3:1 as developing agent, 175 mg of white solid was obtained with a yield after separation of 81%.

Characterization data of 6-trifluoromethylbenzothiazol-2-one: ¹H NMR (DMSO-d 6,500 MHz): δ (ppm) 12.22 (brs, 1H), 7.85 (d, 1H, J=8.5 Hz), 7.48 (dd, 1H, J1=8.0 Hz, J2=1.0 Hz), 7.33 (d, 1H, J=1.5 Hz); 13C NMR (DMSO-d 6, 125 MHz): δ (ppm) 169.7, 136.7, 128.4 (d, J=1.25 Hz), 126.9 (q, J=31.9.5 Hz), 124.0 (q, J=270.5 Hz), 123.8, 119.0 (q, J=3.9 Hz), 107.6 (q, J=4.1 Hz); MS (EI): M/z calcd for C8H4F3NOS [M+1]+: 220.0, found 219.0. m.p .: 216-218° C.

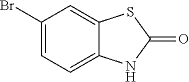 [SMILES: O=c1[nH]c2ccc(Br)cc2s1]

Separated by dry packing and dry sample loading column chromatography (200-300 mesh silica gel): dichloromethane:ethyl acetate (V/V)=20:1 as developing agent, 144 mg of white solid was obtained with a yield after separation of 63%.

Characterization data of 6-bromobenzothiazol-2-one: ¹H NMR (DMSO-d₆, 500 MHZ): δ (ppm) 12.02 (brs, 1H), 7.86 (d, 1H, J=2.0 Hz), 7.44 (dd, 1H, J1=8.5, J2=2.5 Hz), 7.05 (d, 1H, J=8.5 Hz); ¹³C NMR (DMSO-d₆, 125 MHz): δ (ppm) 169.7, 135.6, 129.2, 125.6, 125.0, 114.0, 113.1; MS (EI): m/z calcd for C7H4BrNOS [M+1]+: 229.9, found 228.9. m.p. : 231-232° C.

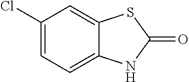 [SMILES: O=c1[nH]c2ccc(Cl)cc2s1]

Separated by dry packing and dry sample loading column chromatography (200-300 mesh silica gel): dichloromethane:ethyl acetate (V/V)=100:3 as developing agent, 121 mg of white solid was obtained with a yield after separation of 66%.

Characterization data of 6-chlorobenzothiazol-2-one: ¹H NMR (DMSO-d₆, 500 MHz): δ (ppm) 12.02 (brs, 1H), 7.74 (d, 1H, J=2.0 Hz), 7.32 (dd, 1H, J1=8.5, J2=2.5 Hz), 7.11 (d, 1H, J=8.5 Hz); ¹³C NMR (DMSO-d₆, 125 MHz): δ (ppm) 169.7, 135.3, 126.4, 125.2, 122.4, 122.7; MS (ESI): m/z calcd for C7H4ClNOS [M+1]+: 186.0, found 185.0. m.p. : 212-214° C.

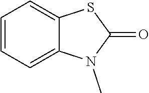

Separated by dry packing and dry sample loading column chromatography (200-300 mesh silica gel): dichloromethane:ethyl acetate (V/V)=100:3 as developing agent, 124 mg of white solid was obtained with a yield after separation of 76%.

Characterization data of 2-methylbenzothiazole -2-one: ¹H NMR (500 MHz, DMSO-d₆) δ (ppm) 7.64 (d, J=7.8 Hz, 1H), 7.39 (t, J=7.7 Hz, 1H), 7.30 (d, J=8.1 Hz, 1H), 7.21 (t, J=7.6 Hz, 1H), 3.41 (s, 3H). ¹³C NMR (126 MHz, DMSO-d₆) δ (ppm)137.61, 126.60, 123.17, 122.70, 121.28, 111.32.

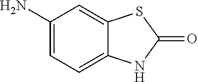 [SMILES: Nc1ccc2[nH]c(=O)sc2c1]

Separated by dry packing and dry sample loading column chromatography (200-300 mesh silica gel): petroleum ether:ethyl acetate (V/V)=3:1 as developing agent, 110 mg of white solid was obtained with a yield after separation of 67%.

Characterization data of 6-aminobenzothiazole-2-one: ¹H NMR (500 MHz, DMSO-d6) δ (ppm) 11.34 (s, 1H), 6.80 (d, J=8.4 Hz, 1H), 6.69 (d, J=2.2 Hz, 1H), 6.51 (dd, J=8.4, 2.3 Hz, 1H), 4.94 (s, 2H). 13C NMR (126 MHz, DMSO-d6) δ (ppm) 169.31, 144.70, 126.35, 124.07, 112.92, 111.98, 107.03. MS (ESI): m/z calcd for C7H6N2OS [M+1]+: 167.1, found 166.1.

### Example 2 Synthesis of Benzothiazolone Derivatives by the Reaction of o-Nitroiodobenzene with CO₂ and H₂S

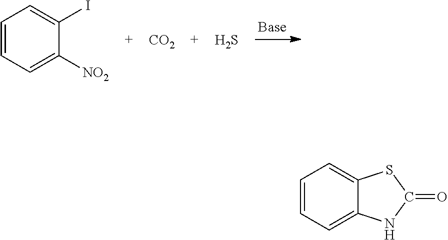

The reaction method was as follows:

1 mmol of o-halogenated nitrobenzene, 2 mmol of base, 0.2 mmol of cuprous iodide (CuI) and 2 ml of solvent were weighed and added to the reactor sequentially and the reactor was tightened. The corresponding amount of H₂S was introduced into the reactor, and the reaction was stirred at the corresponding temperature for 30 min, then the corresponding amount of CO₂ was introduced into the reactor, and the reaction was continued to be stirred at the corresponding temperature for 24 h. After the reaction, the reactor was cooled to room temperature, and the reactor was opened after the gas in the reactor was slowly exhausted, then the reaction mixture was transferred to a 250 ml partition funnel and extracted with ethyl acetate. The organic phase was dried with anhydrous magnesium sulfate, and separated by column chromatography to obtain the product.

The conditions were optimized according to the above steps, and the reaction results were shown in the following table:

| | | | | | o-iodine | P(MPa) | | |
|---|---|---|---|---|---|---|---|---|
| Entry | base | Additive | Solvent | T/° C. | nitrobenzene:base | CO₂ | H₂S | Yield (%) |
| 1 | DBU | — | NMP | 90 | 1:3 | 3 | 0.5 | 0 |
| 2 | DBU | — | NMP/H₂O | 90 | 1:3 | 3 | 0.5 | 0 |
| 3 | Et₃N | — | NMP | 90 | 1:3 | 3 | 0.5 | 31 |
| 4 | Et₃N | — | NMP/H₂O | 90 | 1:3 | 3 | 1 | 32 |
| 5 | Et₃N | — | H₂O | 90 | 1:3 | 3 | 1 | 0 |
| 6 | Et₃N | — | NMP | 90 | 1:3 | 3 | 1 | 32 |
| 7 | DBU | CuI | NMP | 90 | 1:3 | 3 | 1 | 34 |
| 8 | Et₃N | CuI | NMP | 90 | 1:2 | 3 | 1 | 75 |
| 9 | Et₃N | CuI | NMP | 90 | 1:3 | 3 | 1 | 90 |
| 10 | Et₃N | CuI | NMP | 90 | 1:4 | 3 | 1 | 86 |
| 11 | Et₃N | CuI | NMP | 80 | 1:3 | 3 | 1 | 76 |

Note:
in each of the above entries, the raw material was 1 mmol o-iodonitrobenzene; the solvent used was 2 ml; CuI used was 0.2 mmol; and was reacted for 24 h.

### Using the Method as in Entry 9 Above While Changing Other Reaction Substrates, the Following Individual Products were Obtained

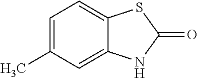

Separated by dry packing and dry sample loading column chromatography (200-300 mesh silica gel): petroleum ether:ethyl acetate (V/V)=3:1 as developing agent, 117 mg of white solid was obtained with a yield after separation of 71%.

Characterization data of 5-methylbenzothiazole-2-one: ¹H NMR (500 MHz, DMSO-d₆) δ (ppm) 11.80 (s, 1H), 7.42 (d, J=7.9 Hz, 1H), 6.97-6.92 (m, 2H), 2.32 (s, 3H). ¹³C NMR (126 MHz, DMSO-d₆) δ (ppm)170.35, 136.35, 136.06, 123.52, 122.39, 119.97, 111.87, 21.00; MS (ESI): m/z calcd for C8H7NOS [M+1]+: 166.0, found 165.0.

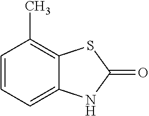

Separated by dry packing and dry sample loading column chromatography (200-300 mesh silica gel): petroleum ether:ethyl acetate (V/V)=3:1 as developing agent, 117 mg of white solid was obtained with a yield after separation of 71%.

Characterization data of 7-methylbenzothiazole-2-one: 1H NMR (500 MHz, DMSO-d6) δ 11.87 (s, 1H), 7.20 (t, J=7.8 Hz, 1H), 6.97 (dd, J=7.7, 5.0 Hz, 2H), 2.28 (s, 3H). 13C NMR (126 MHz, DMSO-d6) δ (ppm) 169.57, 136.09, 131.62, 126.25, 123.07, 122.98, 109.03, 19.70; MS (ESI): m/z calcd for C8H7NOS [M+1]+: 166.0, found 165.0.

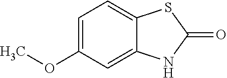

Separated by dry packing and dry sample loading column chromatography (200-300 mesh silica gel): petroleum ether:ethyl acetate (V/V)=3:1 as developing agent, 127 mg of white solid was obtained with a yield after separation of 70%.

Characterization data of 5-methoxybenzothiazole-2-one: ¹H NMR (500 MHz, DMSO-d6) δ (ppm)11.80 (s, 1H), 7.44 (d, J=8.7 Hz, 1H), 6.74 (dd, J=8.7, 2.5 Hz, 1H), 6.66 (d, J=2.5 Hz, 1H), 3.75 (s, 3H). ¹³C NMR (126 MHz, DMSO-d6) δ (ppm)170.91, 158.48, 137.27, 123.40, 114.19, 109.48, 97.39, 55.39; MS (ESI): m/z calcd for C8H7NO2S [M+1]+: 182.1, found 181.1.

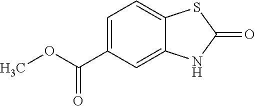

Separated by dry packing and dry sample loading column chromatography (200-300 mesh silica gel): petroleum ether:ethyl acetate (V/V)=3:1 as developing agent, 140 mg of white solid was obtained with a yield after separation of 67%.

Characterization data of 5-methyl ester benzothiazole-2-one: ¹H NMR (500 MHz, DMSO-d6) δ (ppm) 12.14 (s, 1H), 7.75-7.68 (m, 3H), 7.63 (s, 1H), 3.87 (s, 4H). ¹³C NMR (126 MHz, DMSO-d6) δ (ppm) 169.69, 165.75, 136.52, 129.31, 127.63, 123.14, 122.96, 111.50, 111.46, 52.29. MS (ESI): m/z calcd for C9H7NO3S [M+1]+: 220.0, found 219.0.

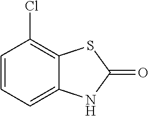 [SMILES: O=c1[nH]c2cccc(Cl)c2s1]

Separated by dry packing and dry sample loading column chromatography (200-300 mesh silica gel): petroleum ether:ethyl acetate (V/V)=5:1 as developing agent, 116 mg of white solid was obtained with a yield after separation of 63%.

Characterization data of 7-chlorobenzothiazole-2-one: ¹H NMR (500 MHz, DMSO-d6) δ (ppm) 12.22 (s, 1H), 7.33 (t, J=8.0 Hz, 1H), 7.26 (d, J=7.1 Hz, 1H), 7.11 (d, J=7.9 Hz, 1H). ¹³C NMR (126 MHz, DMSO-d6) δ (ppm) 168.56, 137.50, 127.83, 126.19, 122.61, 122.19, 110.34, 110.30, 109.54; MS (ESI): m/z calcd for C7H4ClNOS [M+1]+: 186.0, found 185.0.

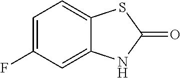 [SMILES: O=c1[nH]c2cc(F)ccc2s1]

Separated by dry packing and dry sample loading column chromatography (200-300 mesh silica gel): petroleum ether:ethyl acetate (V/V)=3:1 as developing agent, 126 mg of white solid was obtained with a yield after separation of 75%.

Characterization data of 5-fluorobenzothiazole-2-one: ¹H NMR (500 MHz, DMSO-d₆) δ (ppm) 12.02 (s, 1H), 7.60 (dd, J=8.7, 5.4 Hz, 1H), 7.00 (td, J=9.1, 2.6 Hz, 1H), 6.93 (dd, J=9.3, 2.6 Hz, 1H). ¹³C NMR (DMSO-d₆, 126 MHz) δ (ppm) 170.68, 161.02 (d, J=57.9 Hz), 137.31 (d, J=6.1 Hz), 124.17 (d, J=4.8 Hz), 118.72 (d, J=1.2 Hz), 109.74 (d, J=11.7 Hz), 99.22 (d, J=13.7 Hz). MS (EI): m/z calcd for C7H5NOS [M]+: 169.0, found 169.0. m.p.: 172-174° C.

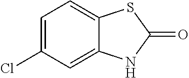 [SMILES: O=c1[nH]c2cc(Cl)ccc2s1]

Separated by dry packing and dry sample loading column chromatography (200-300 mesh silica gel): petroleum ether:ethyl acetate (V/V)=3:1 as developing agent, 118 mg of white solid was obtained with a yield after separation of 64%.

Characterization data of 5-chlorobenzothiazole-2-one: ¹H NMR (500 MHz, DMSO-d6) δ (ppm) 12.05 (s, 1H), 7.61 (d, J=8.4 Hz, 1H), 7.19 (dd, J=8.4, 2.1 Hz, 1H), 7.12 (s, 1H). ¹³C NMR (126 MHz, DMSO-d6) δ (ppm) 170.12, 137.47, 130.83, 124.26, 122.45, 122.22, 122.19, 111.24; MS (ESI): m/z calcd for C7H4ClNOS [m+1]+: 186.0, found 185.0. m.p.: 224-226° C.

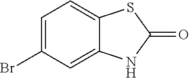 [SMILES: O=c1[nH]c2cc(Br)ccc2s1]

Separated by dry packing and dry sample loading column chromatography (200-300 mesh silica gel): petroleum ether:ethyl acetate (V/V)=3:1 as developing agent, 154 mg of white solid was obtained with a yield after separation of 67%.

Characterization data of 5-bromobenzothiazol-2-one: ¹H NMR (500 MHz, DMSO-d6) δ (ppm) 12.04 (s, 1H), 7.56 (d, J=8.4 Hz, 1H), 7.31 (dd, J=8.4, 2.0 Hz, 1H), 7.24 (d, J=1.9 Hz, 1H). ¹³C NMR (126 MHz, DMSO-d6) δ (ppm) 169.91, 137.70, 125.20, 124.59, 122.71, 118.74, 113.96; MS (ESI): m/z calcd for C7H4BrNOS [M+1]+: 229.9, found 228.9.

### Example 3 Synthesis of Thiazolidine-2-One Derivatives by Reaction of Propargylamine with CO₂ and H₂S

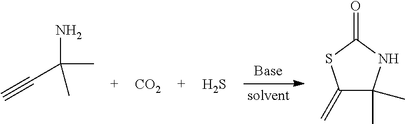

2 mmol of propargylamine, 1.2 mmol of base and 2 mL of solvent were added into a 10 mL reactor, a magnet was placed and the reactor was tightened. After the air in the reactor was replaced with N₂ gas for three times, the corresponding amount of H₂S was introduced, and stirred until the pressure was stabilized. 1 MPa CO₂ was introduced, and the reaction was stirred at the corresponding temperature for 24 h. The reaction mixture was extracted with ethyl acetate after the reaction was completed, then the organic phase was collected and combined, dried with anhydrous magnesium sulfate for 30 min, filtered to remove the desiccant, and the solvent was removed under reduced pressure to obtain the crude product. The crude product was purified by column chromatography (eluent: petroleum ether/ethyl acetate or dichloromethane/methanol) to obtain the target product.

The conditions were optimized according to the above steps, and the results are shown in the following table:

| | base | | P(MPa) | | | Yield |
|---|---|---|---|---|---|---|
| Entry | (equiv.) | Solvent | H₂S | CO₂ | T(° C.) | (%) |
| 1 | DBU(1.0) | DMSO | 1 | 1 | 60 | 68 |
| 2 | DBU(1.0) | DMSO | 1 | 1 | 25 | 81 |
| 3 | DBU(0.6) | DMSO | 1 | 1 | 25 | 81 |
| 4 | Et₃N(0.6) | DMSO | 1 | 1 | 25 | 71 |
| 5 | TBD(0.6) | DMSO | 1 | 1 | 25 | 80 |
| 6 | K₂CO₃(0.6) | DMSO | 1 | 1 | 25 | 39 |

Note:
the raw material is 2 mmol: 2-methyl -3 butyne -2-amine; the solvent are 2 ml; and reacted for 24 hours.

### Using the Method as in Entry 3 Above While Changing Other Reaction Substrates, the Following Individual Products were Obtained

### Characterization of Compounds

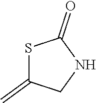

Separated by wet packing and dry sample loading column chromatography (200-300 mesh silica gel): petroleum ether:ethyl acetate (V/V)=1:2 as developing agent, the yield after separation was 24%.

5-methylenethiazolidin-2-one: ¹H NMR (500 MHz, CDCl₃) δ 6.53 (s, 1H), 5.23 (q, J=2.2 Hz, 1H), 5.18-5.11 (m, 1H), 4.31 (s, 2H) ppm; ¹³C NMR (125 MHz, CDCl₃) δ 173.1, 138.8, 106.9, 49.4 ppm; HRMS(ESI) m/z:[M+H]⁺ Calcd for C₄H₅NOS 116.0165; Found 116.0167.

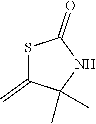

Separated by wet packing and dry sample loading column chromatography (200-300 mesh silica gel): petroleum ether:ethyl acetate (V/V)=2:1 as developing agent, the yield after separation was 81%.

4,4-dimethyl-5-methylenethiazolidin-2-one: ¹H NMR (500 MHz, CDCl₃) δ 6.32 (s, 1H), 5.19 (d, J=2.2 Hz, 1H), 5.11-5.06 (m, 1H), 1.50 (s, 6H) ppm; ¹³C NMR (125 MHz, CDCl₃) δ 169.7, 149.3, 104.9, 62.7, 29.9 ppm; HRMS(ESI) m/z: [M+H]⁺ Calcd for C₆H₉NOS 144.0478; Found 144.0474

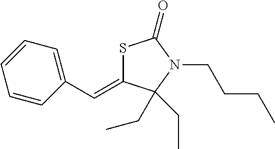 [SMILES: CCCCN1C(=O)S/C(=C\c2ccccc2)C1(CC)CC]

Separated by wet packing and dry sample loading column chromatography (200-300 mesh silica gel): dichloromethane:methanol (V/V)=200:1 as developing agent, the yield after separation was 80%.

(Z)-5-benzylidene-3-butyl-4,4-diethylthiazolidin-2-one:¹H NMR (500 MHz, CDCl₃) δ 7.40-7.31 (m, 4H), 7.25-7.21 (m, 1H), 6.39 (s, 1H), 3.21-3.14 (m, 2H), 1.95-1.83 (m, 2H), 1.78-1.69 (m, 2H), 1.69-1.63 (m, 2H), 1.41-1.32 (m, 2H), 0.96 (t, J=7.4 Hz, 3H), 0.86 (t, J=7.2 Hz, 6H) ppm; ¹³C NMR (125 MHz, CDCl₃) δ 168.8, 136.4, 136.3, 128.8, 128.1, 127.1, 118.2, 75.9, 42.6, 34.1, 31.0, 20.8, 13.9, 7.8 ppm; HRMS(ESI) m/z:[M+H]⁺ Calcd for C₁₈H₂₅NOS 304.1730; Found 304.1725.

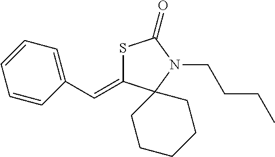 [SMILES: CCCCN1C(=O)S/C(=C\c2ccccc2)C12CCCCC2]

Separated by wet packing and dry sample loading column chromatography (200-300 mesh silica gel): petroleum ether:ethyl acetate (V/V)=2:1 as developing agent, the yield after separation was 92%.

(Z)-4-benzylidene-1-butyl-3-thia-1-azaspiro[4.5]decan-2-one: ¹H NMR (500 MHz, CDCl₃) δ 7.40-7.30 (m, 4H), 7.28-7.22 (m, 1H), 6.96 (s, 1H), 3.29-3.21 (m, 2H), 2.07 (d, J=10.4 Hz, 2H), 1.87-1.72 (m, 7H), 1.63-1.50 (m, 2H), 1.40-1.25 (m, 3H), 0.94 (t, J=7.4 Hz, 3H) ppm; ¹³C NMR (125 MHz, CDCl₃) δ 168.1, 139.6, 136.3, 128.6, 128.5, 127.5, 122.7, 69.7, 42.4, 33.7, 32.2, 24.7, 22.8, 20.5, 13.9 ppm; HRMS(ESI) m/z:[M+H]⁺ Calcd for C₁₉H₂₅NOS 316.1730; Found 316.1722.

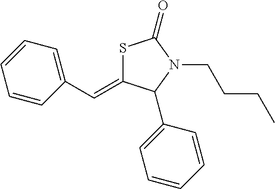 [SMILES: CCCCN1C(=O)S/C(=C\c2ccccc2)C1c1ccccc1]

Separated by wet packing and dry sample loading column chromatography (200-300 mesh silica gel): petroleum ether:ethyl acetate (V/V)=3:1 as developing agent, the yield after separation was 92%.

(Z)-5-benzylidene-3-butyl-4-phenylthiazolidin-2-one: According to general procedure, the crude residue was purified by flash chromatography (PE/EA=3/1) to give the product as a yellow solid (589 mg, 91%). m.p.=83-86° C.; ¹H NMR (500 MHz, CDCl₃) δ 7.45-7.37 (m, 5H), 7.35-7.30 (m, 2H), 7.25-7.19 (m, 3H), 6.31 (s, 1H), 5.48 (s, 1H), 3.76-3.65 (m, 1H), 2.77-2.65 (m, 1H), 1.53-1.42 (m, 2H), 1.33-1.23 (m, 2H), 0.88 (t, J=7.4 Hz, 3H) ppm; ¹³C NMR (125 MHz, CDCl₃) δ 168.0, 139.4, 135.7, 132.4, 129.4, 129.1, 128.7, 128.1, 127.4, 123.3, 69.9, 42.9, 29.2, 20.0, 13.8 ppm; HRMS(ESI) m/z:[M+H]+Calcd for C₂₀H₂₁NOS 324.1417; Found 324.1409.

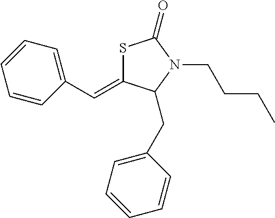 [SMILES: CCCCN1C(=O)S/C(=C\c2ccccc2)C1Cc1ccccc1]

Separated by wet packing and dry sample loading column chromatography (200-300 mesh silica gel): petroleum ether:ethyl acetate (V/V)=2:1 as developing agent, the yield after separation was 99%.

(Z)-4-benzyl-5-benzylidene-3-butylthiazolidin-2-one: ¹H NMR (500 MHz, CDCl₃) δ 7.33 (t, J=7.6 Hz, 2H), 7.29-7.20 (m, 4H), 7.19-7.11 (m, 4H), 6.07 (s, 1H), 4.69-4.49 (m, 1H), 3.92 (dt, J=14.5, 8.0 Hz, 1H), 3.13 (dd, J=13.6, 4.1 Hz, 1H), 3.10-2.98 (m, 2H), 1.69-1.55 (m, 2H), 1.41-1.29 (m, 2H), 0.95 (t, J=7.4 Hz, 3H) ppm; ¹³C NMR (125 MHz, CDCl₃) δ 168.1, 135.4, 135.2, 131.0, 129.9, 128.6, 128.5, 127.9, 127.3, 127.1, 122.4, 66.6, 42.5, 40.5, 29.6, 20.0, 13.7 ppm; HRMS(ESI) m/z:[M+H]⁺ Calcd for C₂₁H₂₃NOS 338.1573; Found 338.1542.

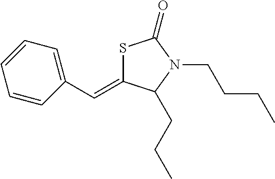 [SMILES: CCCCN1C(=O)S/C(=C\c2ccccc2)C1CCC]

Separated by wet packing and dry sample loading column chromatography (200-300 mesh silica gel): petroleum ether:ethyl acetate (V/V)=2:1 as developing agent, the yield after separation was 90%.

(Z)-5-benzylidene-3-butyl-4-propylthiazolidin-2-one: ¹H NMR (500 MHz, CDCl₃) δ 7.37 (t, J=7.7 Hz, 2H), 7.31 (d, J=7.3 Hz, 2H), 7.23 (d, J=7.3 Hz, 1H), 6.49 (s, 1H), 4.56 (s, 1H), 3.85-3.74 (m, 1H), 3.05-2.93 (m, 1H), 1.97-1.87 (m, 1H), 1.79-1.68 (m, 1H), 1.66-1.49 (m, 2H), 1.47-1.30 (m, 4H), 0.95 (t, J=7.4 Hz, 6H) ppm; ¹³C NMR (125 MHz, CDCl₃) δ 168.1, 135.8, 132.5, 128.8, 128.1, 127.3, 121.1, 65.2, 42.3, 36.4, 29.6, 20.1, 16.1, 14.1, 13.9 ppm; HRMS(ESI) m/z:[M+H]⁺ Calcd for C₁₇H₂₃NOS 290.1573; Found 290.1558.

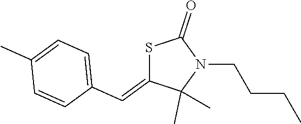 [SMILES: CCCCN1C(=O)S/C(=C\c2ccc(C)cc2)C1(C)C]

Separated by wet packing and dry sample loading column chromatography (200-300 mesh silica gel): petroleum ether:ethyl acetate (V/V)=2:1 as developing agent, the yield after separation was 92%.

(Z)-3-butyl-4,4-dimethyl-5-(4-methylbenzylidene)thiazolidin-2-one: ¹H NMR (500 MHz, CDCl₃) δ 7.23 (d, J=8.2 Hz, 2H), 7.17 (d, J=8.0 Hz, 2H), 6.50 (s, 1H), 3.29-3.23 (m, 2H), 2.34 (s, 3H), 1.68-1.59 (m, 2H), 1.55 (s, 6H), 1.41-1.31 (m, 2H), 0.95 (t, J=7.4 Hz, 3H) ppm; ¹³C NMR (125 MHz, CDCl₃) δ 167.1, 138.3, 137.1, 133.3, 129.4, 128.1, 119.0, 68.0, 42.5, 31.7, 28.3, 21.3, 20.5, 13.9 ppm; HRMS(ESI) m/z: [M+H]⁺ Calcd for C₁₇H₂₃NOS 290.1573; Found 290.1568.

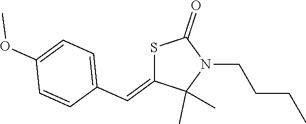 [SMILES: CCCCN1C(=O)S/C(=C\c2ccc(OC)cc2)C1(C)C]

Separated by wet packing and dry sample loading column chromatography (200-300 mesh silica gel): petroleum ether:ethyl acetate (V/V)=2:1 as developing agent, the yield after separation was 81%.

(Z)-3-butyl-5-(4-methoxybenzylidene)-4,4-dimethylthiazolidin-2-one: ¹H NMR (500 MHz, CDCl₃) δ 7.27 (d, J=10.5 Hz, 2H), 6.90 (d, J=8.8 Hz, 2H), 6.47 (s, 1H), 3.82 (s, 3H), 3.29-3.22 (m, 2H), 1.68-1.60 (m, 2H), 1.54 (s, 6H), 1.40-1.31 (m, 2H), 0.95 (t, J=7.4 Hz, 3H) ppm; ¹³C NMR (125 MHz, CDCl₃) δ 167.2, 158.7, 136.9, 129.5, 128.9, 118.6, 114.2, 68.0, 55.4, 42.5, 31.7, 28.3, 20.5, 13.9 ppm; HRMS(ESI) m/z: [M+H]⁺ Calcd for C₁₇H₂₃NO₂S 306.1522; Found 306.1516.

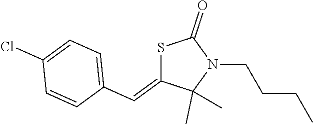 [SMILES: CCCCN1C(=O)S/C(=C\c2ccc(Cl)cc2)C1(C)C]

Separated by wet packing and dry sample loading column chromatography (200-300 mesh silica gel): petroleum ether:ethyl acetate (V/V)=2:1 as developing agent, the yield after separation was 83%.

(Z)-3-butyl-5-(4-chlorobenzylidene)-4,4-dimethylthiazolidin-2-one: ¹H NMR (500 MHz, CDCl₃) δ 7.33 (d, J=8.5 Hz, 2H), 7.26 (d, J=8.7 Hz, 2H), 6.47 (s, 1H), 3.30-3.23 (m, 2H), 1.68 - 1.60 (m, 2H), 1.55 (s, 6H), 1.42 - 1.31 (m, 2H), 0.95 (t, J=7.3 Hz, 3H) ppm; ¹³C NMR (125 MHz, CDCl₃) δ 166.5, 140.5, 134.7, 132.8, 129.4, 128.9, 117.8, 68.1, 42.6, 31.7, 28.3, 20.5, 13.9 ppm; HRMS(ESI) m/z: [M+H]⁺ Calcd for C₁₆H₂₀ClNOS 310.1027; Found 310.1021.

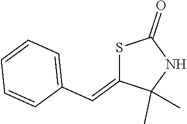

Separated by wet packing and dry sample loading column chromatography (200-300 mesh silica gel): petroleum ether:ethyl acetate (V/V)=1:2 as developing agent, the yield after separation was 87%.

(Z)-5-benzylidene-4.4-dimethylthiazolidin-2-one: ¹H NMR (500 MHz, DMSO-d₆) δ 8.79 (s, 1H), 7.43-7.33 (m, 4H), 7.25 (t, J=7.3 Hz, 1H), 6.74 (s, 1H), 1.50 (s, 6H) ppm; ¹³C NMR (125 MHz, DMSO-d₆) δ 166.1, 140.8, 136.0, 128.6, 127.7, 126.9, 118.7, 63.5, 29.7 ppm; HRMS(ESI) m/z: [M+H]⁺ Calcd for C₁₂H₁₃NOS 220.0791; Found 220.0788.

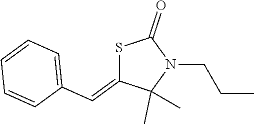 [SMILES: CCCN1C(=O)S/C(=C\c2ccccc2)C1(C)C]

Separated by wet packing and dry sample loading column chromatography (200-300 mesh silica gel): petroleum ether:ethyl acetate (V/V)=2:1 as developing agent, the yield after separation was 94%.

(Z)-5-benzylidene-4,4-dimethyl-3-propylthiazolidin-2-one: ¹H NMR (500 MHz, CDCl₃) 1 δ 7.41-7.29 (m, 4H), 7.25-7.21 (m, 1H), 6.53 (s, 1H), 3.27-3.16 (m, 2H), 1.74-1.63 (m, 2H), 1.56 (s, 6H), 0.94 (t, J=7.4 Hz, 3H) ppm; ¹³C NMR (125 MHz, CDCl₃) δ 166.9, 139.3, 136.0, 128.6, 128.0, 127.1, 118.9, 67.9, 44.2, 28.2, 22.7, 11.5 ppm; HRMS(ESI) m/z: [M+H]⁺ Calcd for C₁₅H₁₉NOS 262.1260; Found 262.1257.

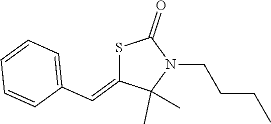 [SMILES: CCCCN1C(=O)S/C(=C\c2ccccc2)C1(C)C]

Separated by wet packing and dry sample loading column chromatography (200-300 mesh silica gel): petroleum ether:ethyl acetate (V/V)=2:1 as developing agent, the yield after separation was 87%.

(Z)-5-benzylidene-3-butyl-4,4-dimethylthiazolidin-2-one: ¹H NMR (500 MHz, CDCl₃) δ 7.39-7.31 (m, 4H), 7.25-7.21 (m, 1H), 6.53 (s, 1H), 3.30-3.24 (m, 2H), 1.69-1.60 (m, 2H), 1.56 (s, 6H), 1.41-1.32 (m, 2H), 0.95 (t, J=7.4 Hz, 3H) ppm; ¹³C NMR (125 MHz, CDCl₃) δ 166.9, 139.6, 136.2, 128.7, 128.2, 127.3, 119.0, 68.1, 42.5, 31.7, 28.4, 20.6, 13.9 ppm; HRMS(ESI) m/z: [M+H]⁺ Calcd for C₁₆H₂₁NOS 276.1417; Found 276.1416.

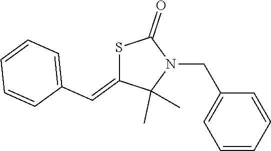 [SMILES: CC1(C)/C(=C/c2ccccc2)SC(=O)N1Cc1ccccc1]

Separated by wet packing and dry sample loading column chromatography (200-300 mesh silica gel): petroleum ether:ethyl acetate (V/V)=2:1 as developing agent, the yield after separation was 89%.

(Z)-3-benzyl-5-benzylidene-4,4-dimethylthiazolidin-2-one: ¹H NMR (500 MHz, CDCl₃) δ 7.40-7.33 (m, 4H), 7.32-7.29 (m, 4H), 7.27-7.23 (m, 2H), 6.53 (s, 1H), 4.62 (s, 2H), 1.49 (s, 6H) ppm; ¹³C NMR (125 MHz, CDCl₃) δ 168.1, 139.1, 138.1, 136.0, 128.8, 128.7, 128.2, 127.5, 127.4, 127.3, 119.3, 68.4, 45.3, 28.5 ppm; HRMS(ESI) m/z: [M+H]⁺ Calcd for C₁₉H₁₉NOS 310.1260; Found 310.1250.

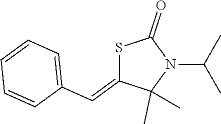 [SMILES: CC(C)N1C(=O)S/C(=C\c2ccccc2)C1(C)C]

Separated by wet packing and dry sample loading column chromatography (200-300 mesh silica gel): petroleum ether:ethyl acetate (V/V)=4:1 as developing agent, the yield after separation was 46%.

(Z)-5-benzylidene-3-isopropyl-4,4-dimethylthiazolidin-2-one: ¹H NMR (500 MHz, CDCl₃) δ 7.40-7.31 (m, 4H), 7.25-7.20 (m, 1H), 6.46 (s, 1H), 3.58-3.47 (m, 1H), 1.56 (s, 6H), 1.49 (d, J=6.8 Hz, 6H) ppm; ¹³C NMR (125 MHz, CDCl₃) δ 165.6, 139.8, 136.3, 128.7, 128.2, 127.1, 118.8, 69.1, 47.7, 28.3, 20.5 ppm; HRMS(ESI) m/z: [M+H]⁺ Calcd for C₁₅H₁₉NOS 262.1260; Found 262.1256

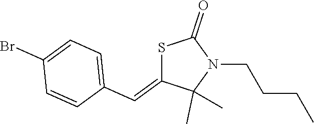 [SMILES: CCCCN1C(=O)S/C(=C\c2ccc(Br)cc2)C1(C)C]

Separated by wet packing and dry sample loading column chromatography (200-300 mesh silica gel): petroleum ether:ethyl acetate (V/V)=2:1 as developing agent, the yield after separation was 75%.

(Z)-5-(4-bromobenzylidene)-3-butyl-4,4-dimethylthiazolidin-2-one: ¹H NMR (500 MHz, CDCl₃) δ 7.49 (d, J=8.5 Hz, 2H), 7.20 (d, J=8.6 Hz, 2H), 6.45 (s, 1H), 3.29-3.24 (m, 2H), 1.68-1.60 (m, 2H), 1.55 (s, 6H), 1.40-1.32 (m, 2H), 0.95 (td, J=7.4, 1.1 Hz, 3H) ppm; ¹³C NMR (125 MHz, CDCl₃) δ 166.5, 140.7, 135.1, 131.9, 129.7, 121.0, 117.9, 68.1, 42.6, 31.7, 28.3, 20.6, 13.9 ppm; MS(ESI) m/z: [M+H]⁺ Calcd for C₁₆H₂₀BrNOS 354.1; Found 354.1.

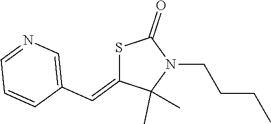 [SMILES: CCCCN1C(=O)S/C(=C\c2cccnc2)C1(C)C]

Separated by wet packing and dry sample loading column chromatography (200-300 mesh silica gel): petroleum ether: ethyl acetate (V/V)=1:3 as developing agent, the yield after separation was 79%.

(Z)-3-butyl-4.4-dimethyl-5-(pyridin-3-ylmethylene)thiazolidin-2-one: ¹H NMR (500 MHz, CDCl₃) δ 8.52 (d, J=55.4 Hz, 2H), 7.70 (d, J=7.5 Hz, 1H), 7.32 (s, 1H), 6.49 (s, 1H), 3.31-3.24 (m, 2H), 1.66-1.63 (m, 2H), 1.58 (s, 6H), 1.41-1.32 (m, 2H), 0.95 (t, J=7.3 Hz, 3H) ppm; ¹³C NMR (125 MHz, CDCl₃) δ 166.1, 150.1, 147.9, 142.9, 134.2, 115.4, 68.3, 42.7, 31.7, 28.4, 20.5, 13.9 ppm; HRMS(ESI) m/z: [M+H]⁺ Calcd for C₁₅H₂₂N₂OS 277.1369; Found 277.1376.

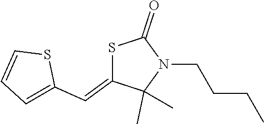 [SMILES: CCCCN1C(=O)S/C(=C\c2cccs2)C1(C)C]

Separated by wet packing and dry sample loading column chromatography (200-300 mesh silica gel): petroleum ether:ethyl acetate (V/V)=4:1 as developing agent, the yield after separation was 82%.

(Z)-3-butyl-4,4-dimethyl-5-(thiophen-2-ylmethylene)thiazolidin-2-one: ¹H NMR (500 MHz, CDCl₃) δ 7.33 (d, J=5.0 Hz, 1H), 7.07-7.04 (m, 1H), 7.02 (d, J=3.1 Hz, 1H), 6.74 (s, 1H), 3.29-3.23 (m, 2H), 1.66-1.61 (m, 2H), 1.54 (s, 6H), 1.40-1.31 (m, 2H), 0.95 (t, J=7.4 Hz, 3H) ppm; ¹³C NMR (125 MHz, CDCl₃) δ 166.4, 140.2, 137.9, 127.5, 126.6, 126.0, 112.0, 67.7, 42.7, 31.7, 28.3, 20.5, 13.9 ppm; HRMS(ESI) m/z: [M+H]⁺ Calcd for C₁₄H₁₉NOS₂ 282.0981; Found 282.0981.

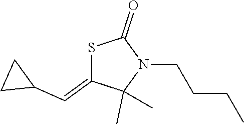 [SMILES: CCCCN1C(=O)S/C(=C\C2CC2)C1(C)C]

Separated by wet packing and dry sample loading column chromatography (200-300 mesh silica gel): petroleum ether:ethyl acetate (V/V)=4:1 as developing agent, the yield after separation was 40%.

(Z)-3-butyl-5-(cyclopropylmethylene)-4,4-dimethylthiazolidin-2-one: ¹H NMR (500 MHz, CDCl₃) δ 4.99 (d, J=8.8 Hz, 1H), 3.22-3.14 (m, 2H), 1.63-1.55 (m, 2H), 1.39 (s, 6H), 1.37-1.28 (m, 3H), 1.23-1.16 (m, 1H), 0.92 (t, J=7.4 Hz, 3H), 0.85-0.78 (m, 2H), 0.44-0.39 (m, 2H) ppm; ¹³C NMR (125 MHz, CDCl₃) δ 167.5, 136.9, 122.9, 66.4, 42.3, 31.8, 28.0, 20.5, 13.9, 13.1, 7.3 ppm; MS(ESI) m/z: [M+H]⁺ Calcd for C₁₃H₂₁NOS 240.1; Found 240.2.

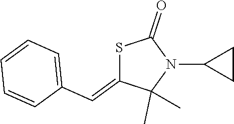 [SMILES: CC1(C)/C(=C/c2ccccc2)SC(=O)N1C1CC1]

Separated by wet packing and dry sample loading column chromatography (200-300 mesh silica gel): petroleum ether:ethyl acetate (V/V)=4:1 as developing agent, the yield after separation was 72%.

(Z)-5-benzylidene-3-cyclopropyl-4.4-dimethylthiazolidin-2-one: ¹H NMR (500 MHz, CDCl₃) δ 7.39-7.31 (m, 4H), 7.25-7.21 (m, 1H), 6.54 (s, 1H), 2.39-2.33 (m, 1H), 1.67 (s, 6H), 0.99-0.94 (m, 2H), 0.93-0.88 (m, 2H) ppm. ¹³C NMR (125 MHz, CDCl₃) δ 168.8, 138.9, 136.1, 128.7, 128.2, 127.3, 119.0, 69.6, 28.3, 24.3, 6.4 ppm; MS(ESI) m/z: [M+H]⁺ Calcd for C₁₅H₁₇NOS 260.1; Found 260.2.

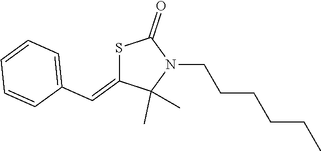 [SMILES: CCCCCCN1C(=O)S/C(=C\c2ccccc2)C1(C)C]

Separated by wet packing and dry sample loading column chromatography (200-300 mesh silica gel): petroleum ether:ethyl acetate (V/V)=4:1 as developing agent, the yield after separation was 85%.

(Z)-5-benzylidene-3-hexyl-4,4-dimethylthiazolidin-2-one: ¹H NMR (500 MHz, CDCl₃) δ 7.39-7.32 (m, 4H), 7.26-7.21 (m, 1H), 6.53 (s, 1H), 3.29-3.22 (m, 2H), 1.69-1.62 (m, 2H), 1.56 (s, 6H), 1.38-1.28 (m, 6H), 0.89 (t, J=6.3 Hz, 3H) ppm; ¹³C NMR (125 MHz, CDCl₃) δ 166.9, 139.5, 136.2, 128.7, 128.2, 127.2, 119.0, 68.1, 42.7, 31.6, 29.6, 28.4, 27.0, 22.72, 14.1 ppm; HRMS(ESI) m/z: [M+H]⁺ Calcd for C₁₈H₂₅NOS 304.1730; Found 304.1734.

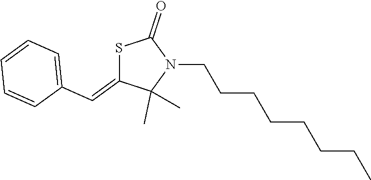 [SMILES: CCCCCCCCN1C(=O)S/C(=C\c2ccccc2)C1(C)C]

Separated by wet packing and dry sample loading column chromatography (200-300 mesh silica gel): petroleum ether:ethyl acetate (V/V)=2:1 as developing agent, the yield after separation was 86%.

(Z)-5-benzylidene-4,4-dimethyl-3-octylthiazolidin-2-one: ¹H NMR (500 MHz, CDCl₃) δ 7.40-7.31 (m, 4H), 7.26-7.21 (m, 1H), 6.53 (s, 1H), 3.28-3.23 (m, 2H), 1.70-1.62 (m, 2H), 1.56 (s, 6H), 1.34-1.23 (m, 10H), 0.88 (t, J=6.7 Hz, 3H) ppm; ¹³C NMR (125 MHz, CDCl₃) δ 166.9, 139.5, 136.2, 128.7, 128.2, 127.3, 119.0, 68.1, 42.8, 31.9, 29.7, 29.3(9), 29.3(7), 28.4, 27.3, 22.8, 14.2 ppm; HRMS(ESI) m/z: [M+H]⁺ Calcd for C₂₀H₂₉NOS 332.2043; Found 332.2049.

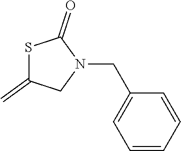

Separated by wet packing and dry sample loading column chromatography (200-300 mesh silica gel): petroleum ether:ethyl acetate (V/V)=5:1 as developing agent, the yield after separation was 35%.

3-benzyl-5-methylenethiazolidin-2-one: ¹H NMR (500 MHz, CDCl₃) δ 7.39-7.34 (m, 2H), 7.33-7.29 (m, 1H), 7.27 (d, J=7.9 Hz, 2H), 5.17-5.14 (m, 1H), 5.14-5.11 (m, 1H), 4.53 (s, 2H), 4.17-4.13 (m, 2H) ppm. ¹³C NMR (125 MHz, CDCl₃) δ 169.1, 135.5, 135.4, 129.0, 128.3, 128.2, 106.6, 53.9, 48.3 ppm; HRMS(ESI) m/z: [M+H]⁺ Calcd for C₄H₅NOS: 206.1; Found 206.0.

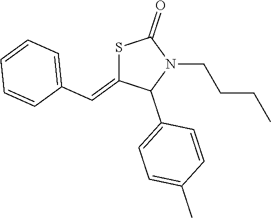 [SMILES: CCCCN1C(=O)S/C(=C\c2ccccc2)C1c1ccc(C)cc1]

Separated by wet packing and dry sample loading column chromatography (200-300 mesh silica gel): petroleum ether:ethyl acetate (V/V)=4:1 as developing agent, the yield after separation was 94%.

(Z)-5-benzylidene-3-hexyl-4-(p-tolyl)thiazolidin-2-one: ¹H NMR (500 MHz, CDCl₃) δ 7.31 (t, J=7.7 Hz, 2H), 7.25 (d, J=3.1 Hz, 2H), 7.24-7.17 (m, 5H), 6.28 (s, 1H), 5.43 (s, 1H), 3.73-3.65 (m, 1H), 2.74-2.66 (m, 1H), 2.37 (s, 3H), 1.51-1.43 (m, 2H), 1.33-1.21 (m, 2H), 0.88 (t, J=7.3 Hz, 3H) ppm; ¹³C NMR (125 MHz, CDCl₃) δ 168.0, 139.0, 136.4, 135.8, 132.7, 130.0, 128.7, 128.1, 127.3(3), 127.3(1), 123.1, 69.7, 42.8, 29.2, 21.4, 20.1, 13.8 ppm; HRMS(ESI) m/z: [M+H]⁺ Calcd for C₂₁H₂₃NOS 338.1573; Found 338.1578.

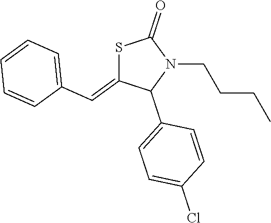 [SMILES: CCCCN1C(=O)S/C(=C\c2ccccc2)C1c1ccc(Cl)cc1]

Separated by wet packing and dry sample loading column chromatography (200-300 mesh silica gel): petroleum ether:ethyl acetate (V/V)=4:1 as developing agent, the yield after separation was 66%.

(Z)-5-benzylidene-3-butyl-4-(4-chlorophenyl)thiazolidin-2-one: ¹H NMR (500 MHz, CDCl₃) δ 7.40 (d, J=8.3 Hz, 2H), 7.33 (d, J=8.2 Hz, 4H), 7.23 (d, J=7.6 Hz, 3H), 6.28 (s, 1H), 5.45 (s, 1H), 3.71 (dt, J=15.4, 7.9 Hz, 1H), 2.73-2.66 (m, 1H), 1.51-1.42 (m, 2H), 1.34-1.21 (m, 2H), 0.89 (t, J=7.3 Hz, 3H) ppm; ¹³C NMR (125 MHz, CDCl₃) δ 168.0, 138.0, 135.5, 135.1, 131.9, 129.7, 128.7, 128.7, 128.1, 127.6, 123.6, 69.1, 42.9, 29.2, 20.0, 13.8 ppm; HRMS(ESI) m/z: [M+H]⁺ Calcd for C₂₀H₂₀ClNOS 358.1027; Found 358.1031.

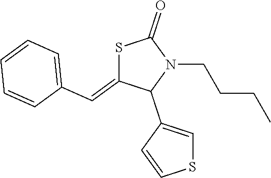 [SMILES: CCCCN1C(=O)S/C(=C\c2ccccc2)C1c1ccsc1]

Separated by wet packing and dry sample loading column chromatography (200-300 mesh silica gel): petroleum ether:ethyl acetate (V/V)=4:1 as developing agent, the yield after separation was 83%.

(Z)-5-benzylidene-3-butyl-4-(thiophen-3-yl)thiazolidin-2-one: ¹H NMR (500 MHz, CDCl₃) δ 7.39-7.31 (m, 4H), 7.22 (dd, J=13.3, 5.9 Hz, 2H), 7.08 (d, J=6.1 Hz, 1H), 6.34 (s, 1H), 5.61 (s, 1H), 3.71-3.63 (m, 1H), 2.84-2.76 (m, 1H), 1.53-1.40 (m, 2H), 1.34-1.22 (m, 3H), 0.89 (t, J=7.3 Hz, 3H) ppm; ¹³C NMR (125 MHz, CDCl₃) δ 167.6, 140.2, 135.7, 131.5, 128.7, 128.1(3), 128.1(2), 127.9, 127.4, 125.8, 124.0, 123.2, 65.4, 42.9, 29.3, 20.1, 13.8 ppm; HRMS(ESI) m/z: [M+H]⁺ Calcd for C₁₈H₁₉NOS₂ 330.0981; Found 330.0981.

### Class II H₂S as Catalyst

### Example 4 Synthesis of Benzimidazolone Derivatives by the Reaction of o-Phenylenediamine and CO₂ in the Presence of H₂S

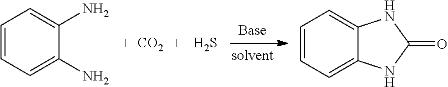

2 mmol of o-phenylenediamine, organic base and 1 mL of suitable solvent were added sequentially into a 15 mL high-pressure reactor, and the reactor was tightened; the required amount of H₂S and CO₂ gas was sequentially introduced into the reactor; finally, the mixture was continuously reacted at a suitable temperature for 12 hours; after the reaction was completed, a certain amount of distilled water was added to the reaction mixture to precipitate the product completely, and then filtered and dried sequentially to obtain the product.

The conditions were optimized according to the above steps, and the results are shown in the following table:

| | | | | | | Yield |
|---|---|---|---|---|---|---|
| Entry | Solvent | base (mmol) | H₂S (P_{MPa}) | CO₂(P_{MPa}) | t (° C.) | (%) |
| 1 | NMP | DBU(3) | 0.08 | 1.5 | 40 | 86 |
| 2 | NMP | DBU(2) | 0.08 | 1.5 | 40 | 86 |
| 3 | NMP | DBU(1) | 0.08 | 1.5 | 40 | 79 |
| 4 | NMP | DBU(2) | 0.3 | 1.5 | 40 | 87 |
| 5 | NMP | DBU(2) | 0.15 | 1.5 | 40 | 87 |
| 6 | NMP | DBU(2) | 0.08 | 1.5 | 40 | 87 |
| 7 | NMP | DBU(2) | 0.03 | 1.5 | 40 | 69 |
| 8 | NMP | DBU(2) | 0 | 1.5 | 40 | NR |
| 9 | NMP | DBU(2) | 0.08 | 5 | 40 | 86 |
| 10 | NMP | DBU(2) | 0.08 | 4 | 40 | 86 |
| 11 | NMP | DBU(2) | 0.08 | 3 | 40 | 87 |
| 12 | NMP | DBU(2) | 0.08 | 1 | 40 | 82 |
| 13 | NMP | DBU(2) | 0.08 | 0 | 40 | NR |
| 14 | NMP | DBU(2) | 0.08 | 1.5 | 60 | 84 |
| 15 | NMP | DBU(2) | 0.08 | 1.5 | 50 | 86 |
| 16 | NMP | DBU(2) | 0.08 | 1.5 | 30 | 85 |
| 17 | NMP | DBU(2) | 0.08 | 1.5 | 20 | 83 |
| 18 | DMSO | DBU(2) | 0.08 | 1.5 | 40 | NR |
| 19 | NMP | DBU(2) | 0.08 | 1.5 | 40 | 87 |
| 20 | NMP | TMG(2) | 0.08 | 1.5 | 40 | 86 |
| 21 | NMP | DBU(2) | 0.08 | 1.5 | 40 | 87 |

Note:
In each of the above entries, the raw material was 2 mmol o-phenylenediamine; and the solvent is 1 ml.

### Using the Method as in Entry 6 while Changing other Reaction Substrates, the Following Products were Obtained

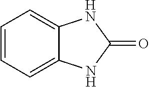

233 mg of white solid product was obtained by filtration and drying, separation yield: 87%

¹H NMR (500 MHz, DMSO-d₆, TMS): δ (ppm) 10.57 (s, 2H), 6.91 (s, 4H).

¹³C NMR (126 MHz, DMSO-d₆, TMS): δ (ppm) 155.28(C), 129.67(C), 120.43(CH), 108.46(CH).

MS (ESI): m/z calcd for C₇H₆NO[M+H]⁺: 135.06, found 135.1.

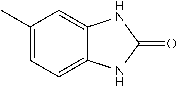

265 mg of solid product was obtained by filtration and drying, separation yield: 89.5%

¹H NMR (500 MHz, DMSO-d₆, TMS): δ (ppm) 10.47 (d, 2H, J=20.0 Hz), 6.80 (d, 1H, J=10.0 Hz), 6.73 (d, 2H, J=10.0 Hz), 2.27 (s, 3H).

¹³C NMR (125 MHz, DMSO-d₆, TMS): δ (ppm) 155.29, 129.72, 129.21, 127.31, 120.75, 108.87, 108.03, 20.91.

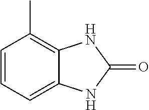

266 mg of solid product, was obtained by filtration and drying, separation yield: 90%

¹H NMR (500 MHz, DMSO-d₆, TMS): δ (ppm) 10.65 (s, 1H), 10.53 (s, 1H), 6.82 (t, 1H, J1=J2=10.0 Hz), 6.74(t, 1H, J1=J2=10.0 Hz), 2.25 (s, 3H).

¹³C NMR (125 MHz, DMSO-d₆, TMS): δ (ppm) 155.47, 129.23, 128.55, 121.56, 120.34, 117.13, 106.05, 16.17.

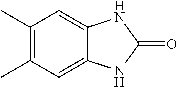

289 mg of solid product, was obtained by filtration and drying, separation yield: 89.2%

¹H NMR (500 MHz, DMSO-d₆, TMS): δ (ppm) 10.34 (s, 2H), 6.70 (s, 2H), 2.17 (s, 6H).

¹³C NMR (125 MHz, DMSO-d₆, TMS): δ (ppm) 155.41, 127.75, 109.54.

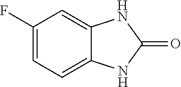 [SMILES: O=c1[nH]c2ccc(F)cc2[nH]1]

176 mg of solid product, was obtained by filtration and drying, separation yield: 58%

¹H NMR (500 MHz, DMSO-d₆, TMS): δ (ppm) 10.74 (s, 1H), 10.63 (s, 1H), 6.87 (dd, J=8.5, 4.7 Hz, 1H), 6.80-6.69 (m, 2H).

¹³C NMR (125 MHz, DMSO-d₆, TMS): δ (ppm) 158.42, 156.13(d, J=110.2 Hz), 130.36(d, J=13.0 Hz), 126.06, 108.77(d, J=9.6 Hz), 106.52(d, J=23.8 Hz), 96.51(d, J=28.2 Hz).

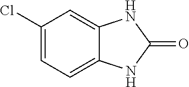 [SMILES: O=c1[nH]c2ccc(Cl)cc2[nH]1]

264 mg of solid product, was obtained by filtration and drying, separation yield: 78%

¹H NMR (500 MHz, DMSO-d₆, TMS): δ (ppm) 10.76 (s, 2H), 6.97-6.90 (m, 3H).

¹³C NMR (125 MHz, DMSO-d₆, TMS): δ (ppm) 155.19, 130.82, 128.60, 124.47, 120.09, 109.50, 108.36.

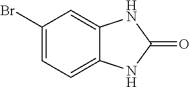 [SMILES: O=c1[nH]c2ccc(Br)cc2[nH]1]

421 mg of solid product, was obtained by filtration and drying, separation yield: 98.7%

¹H NMR (500 MHz, DMSO-d₆, TMS): δ (ppm) 10.76 (s, 2H), 7.05 (t, 2H, J1=J2=10.0 Hz), 6.88 (d, 1H, J=10.0 Hz).

¹³C NMR (125 MHz, DMSO-d₆, TMS): δ (ppm) 155.03, 131.20, 128.98, 122.89, 111.98, 111.02, 110.06.

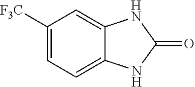 [SMILES: O=c1[nH]c2ccc(C(F)(F)F)cc2[nH]1]

255 mg of solid product, was obtained by filtration and drying, separation yield: 63%

¹H NMR (500 MHz, DMSO-d₆, TMS): δ (ppm) 10.99 (s, 2H), 7.28 (dd, J=8.2, 1.7 Hz, 1H), 7.16 (d, J=1.7 Hz, 1H), 7.09 (d, J=8.1 Hz, 1H).

¹³C NMR (125 MHz, DMSO-d₆, TMS): δ (ppm) 155.36, 132.88, 129.85, 124.89(q, J=271.8 Hz), 120.95(q, J=3.2 Hz), 117.91(q, J=4.2 Hz), 108.58, 104.98(q, J=4.0 Hz).

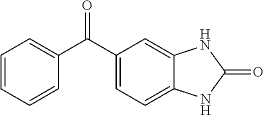 [SMILES: O=C(c1ccccc1)c1ccc2[nH]c(=O)[nH]c2c1]

371 mg of solid product, was obtained by filtration and drying, separation yield: 78%

¹H NMR (500 MHz, DMSO-d₆, TMS): δ (ppm) 11.11 (s, 1H), 10.88 (s, 1H), 7.70-7.63 (m,3H), 7.55 (t, 2H, J1=J2=5.0 Hz), 7.44 (d, 1H, J=10.0 Hz), 7.33 (s, 1H), 7.08 (d, 1H, J=10.0 Hz).

¹³C NMR (125 MHz, DMSO-d₆, TMS): δ (ppm) 194.98, 155.38, 138.19, 134.02, 131.81, 129.66. 129.38. 129.17. 128.34, 124.44, 109.71, 107.98.

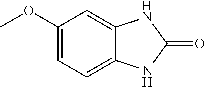

206 mg of solid product, was obtained by filtration and drying, separation yield: 63%

¹H NMR (500 MHz, DMSO-d₆, TMS): δ (ppm) 10.50 (s, 1H), 10.37 (s, 1H), 6.81 (d, 1H, J=5.0 Hz), 6.52 (d, 2H, J=10.0 Hz), 3.70 (s, 3H).

¹³C NMR (125 MHz, DMSO-d₆, TMS): δ (ppm) 155.64, 154.33, 130.49, 123.56, 108.71, 106.07, 95.27, 55.42.

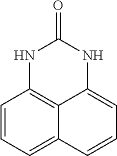 [SMILES: O=C1Nc2cccc3cccc(c23)N1]

358 mg of solid product, was obtained by filtration and drying, separation yield: 97%

¹H NMR (500 MHz, DMSO-d6, TMS): δ (ppm) 10.07 (s, 2H), 7.21 (t, 2H, J1=J2=5.0 Hz), 7.11 (d, 2H, J=5.0 Hz), 6.52 (d, 2H, J=5.0 Hz).

¹³C NMR (125 MHz, DMSO-d6, TMS): δ (ppm) 150.09, 137.67, 134.15, 128.03, 117.65, 113.66, 104.01.

### Example 5 Synthesis of Benzothiazolone Derivatives by Reaction of o-Aminothiophenol and CO₂ in the Presence of H₂S

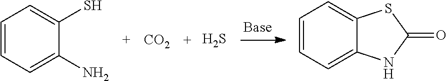

2 mmol of the o-aminothiophenol was placed in a 15 mL stainless steel autoclave equipped with magnet stirrer, and then 2 mmol of base and 2 mL of solvent were added to the autoclave in order, and the autoclave was tightened. The corresponding amount of H₂S was introduced, then the corresponding amount of CO₂ was introduced. And the reaction mixture was stirred vigorously at the reaction temperature for 24 h. Upon completion, the autoclave was cooled down to room temperature and slowly depressurized. The solution was extracted with ethyl acetate, and the organic phases were combined and dried with anhydrous magnesium sulfate. The desiccant was removed by filtration and the solvent was removed under reduced pressure to obtain the crude product, which was purified by column chromatography to obtain the target product.

The conditions were optimized according to the above steps, and the results were shown in the following table:

| | | | molar ratio of o- | P_{CO2} | | | |
|---|---|---|---|---|---|---|---|
| Entry | Solvent | base | aminothiophenol:base | (MPa) | P_{H2S}(MPa) | T(° C.) | Yield (%) |
| 1 | NMP | DBU | 1:1 | 3 | 0 | 40 | trace |
| 2 | NMP | DBU | 1:1 | 3 | 0.2 | 40 | 85.1 |
| 3 | NMP | DBU | 1:1 | 3 | 0.5 | 40 | 89.3 |
| 4 | NMP | DBU | 1:1 | 3 | 1 | 40 | 94.4 |
| 5 | NMP | DBU | 1:0.5 | 3 | 0.2 | 40 | 83.7 |
| 6 | NMP | DBU | 1:1 | 3 | 0.2 | 40 | 90.2 |
| 7 | NMP | DBU | 1:2 | 3 | 0.2 | 40 | 89.7 |
| 8 | NMP | DBU | 1:1 | 4 | 0.2 | 40 | 89.7 |
| 9 | NMP | DBU | 1:1 | 5 | 0.2 | 40 | 91.8 |
| 10 | — | DBU | 1:1 | 8 | 1.0 | 40 | 75.0 |
| 11 | — | DBU | 1:1 | 10 | 1.0 | 40 | 83.0 |
| 12 | — | DBU | 1:1 | 12 | 1.0 | 40 | 77.0 |
| 13 | NMP | DBU | 1:1 | 3 | 0.2 | 20 | 80.0 |
| 14 | NMP | DBU | 1:1 | 3 | 0.2 | 50 | 92.2 |
| 15 | NMP | DBU | 1:1 | 3 | 0.2 | 70 | 89.3 |
| 16 | DMF | DBU | 1:1 | 3 | 0.2 | 40 | 77 |
| 17 | NMP | Et₃N | 1:1 | 3 | 0.2 | 40 | 82.4 |
| 18 | NMP | TMG | 1:1 | 3 | 0.2 | 40 | 90.5 |

Note:
The raw material was 2 mmol o-aminothiophenol; the solvent was2 mL; and the reaction time was 24 hours.

### Using the Method as in Entry 7 while Changing Other Reaction Substrates, the Following Products were Obtained

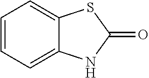

Separated by dry packing and dry sample loading column chromatography (200-300 mesh silica gel): dichloromethane:ethyl acetate (V/V)=20:1 as developing agent, 143 mg of white solid was obtained with a yield after separation of 94.4%.

Characterization data of benzothiazol-2-one (1a):¹H NMR (CDCl₃, 500 MHz): δ (ppm) 10.01 (brs, 1H), 7.41 (d, 1H, J=7.5 Hz), 7.30-7.26 (m, 1H), 7.17-7.14 (m, 2H). ¹³C NMR (CDCl₃, 125 MHz): δ (ppm) 172.8, 135.3, 126.5, 123.9, 123.3, 122.6, 111.7; MS (EI): m/z calcd for C₇H₅NOS [M]⁺: 151.0, found 151.0. m.p.: 139-140° C.

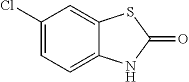 [SMILES: O=c1[nH]c2ccc(Cl)cc2s1]

Separated by dry packing and dry sample loading column chromatography (200-300 mesh silica gel): dichloromethane:ethyl acetate (V/V)=5:1 as developing agent, 141 mg of white solid was obtained with a yield after separation of 76.2%.

Characterization data of 6-chlorobenzothiazole-2-one (1b):¹H NMR (DMSO-d₆, 500 MHz): δ (ppm) 12.02 (brs, 1H), 7.74 (d, 1H, J=2.0Hz), 7.32 (dd, 1H, J₁=8.5, J₂=2.5 Hz), 7.11 (d, 1H, J=8.5 Hz); ¹³C NMR (DMSO-d₆, 125 MHz): δ (ppm) 169.7, 135.3, 126.4, 125.2, 122.4, 122.7; MS (EI): m/z calcd for C₇H₄ClNOS [M]⁺: 185.1, found 185.0. m.p.: 212-214° C.

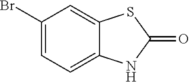 [SMILES: O=c1[nH]c2ccc(Br)cc2s1]

Separated by dry packing and dry sample loading column chromatography (200-300 mesh silica gel): dichloromethane:ethyl acetate (V/V)=20:1 as developing agent, 195 mg of white solid was obtained with a yield after separation of 85%.

Characterization data of 6-bromobenzothiazole-2-one (1c):¹H NMR (DMSO-d₆, 500 MHz): δ (ppm) 12.02 (brs, 1H), 7.86 (d, 1H, J=2.0 Hz), 7.44 (dd, 1H, J₁=8.5, J₂=2.5 Hz), 7.05 (d, 1H, J=8.5 Hz); ¹³C NMR (DMSO-d₆, 125 MHz): δ (ppm) 169.7, 135.6, 129.2, 125.6, 125.0, 114.0, 113.1; MS (EI): m/z calcd for C₇H₄BrNOS [M]⁺: 228.9, found 228.9. m.p.: 231-232° C.

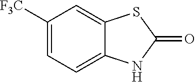 [SMILES: O=c1[nH]c2ccc(C(F)(F)F)cc2s1]

Separated by dry packing and dry sample loading column chromatography (200-300 mesh silica gel): dichloromethane:ethyl acetate (V/V)=20:1 as developing agent, 149 mg of white solid was obtained with a yield after separation of 67.9%.

Characterization data of 6-trifluoromethylbenzothiazole-2-one (1d): ¹H NMR (DMSO-d₆, 500 MHz): δ (ppm) 12.22 (brs, 1H), 7.85 (d, 1H, J=8.5 Hz), 7.48 (dd, 1H, J₁=8.0 Hz, J₂=1.0 Hz), 7.33 (d, 1H, J=1.5 Hz); ¹³C NMR (DMSO-d₆, 125 MHz): δ (ppm) 169.7, 136.7, 128.4 (d, J=1.25 Hz), 126.9 (q, J=31.9.5 Hz), 124.0 (q, J=270.5 Hz), 123.8, 119.0 (q, J=3.9 Hz), 107.6 (q, J=4.1 Hz); MS (EI): m/z calcd for C₈H₄F₃NOS [m]⁺: 219.2, found 219.0. m.p.: 216-218° C.

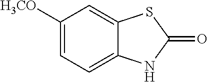 [SMILES: COc1ccc2[nH]c(=O)sc2c1]

Separated by dry packing and dry sample loading column chromatography (200-300 mesh silica gel): dichloromethane:ethyl acetate (V/V)=10:1 as developing agent, 170 mg of white solid was obtained with a yield after separation of 94%.

Characterization data of 6-methoxybenzothiazole-2-one (1e): ¹H NMR (DMSO-d₆, 500 MHz): δ (ppm) 11.658 (brs, 1H), 7.23 (d, 1H, J=2.5 Hz), 7.02 (d, 1H, J=8.5 Hz), 6.86 (dd, 1H, J₁=8.5 Hz, J₂=2.5 Hz), 3.73(s, 3H); ¹³C NMR (DMSO-d₆, 125 MHz): δ (ppm) 169.8, 155.2, 129.9, 124.3, 113.2, 112.1, 107.8, 55.6; MS (EI): M/z calcd for C₈H₇NO₂S [M]⁺: 180.9, found 181.0. m.p.: 161-163° C.

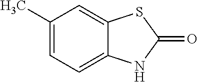

Separated by dry packing and dry sample loading column chromatography (200-300 mesh silica gel): dichloromethane:ethyl acetate (V/V)=50:1 as developing agent, 160 mg of white solid was obtained with a yield after separation of 96.8%.

Characterization data of 6-methylbenzothiazole-2-one:¹H NMR (DMSO-d₆, 500 MHz): δ (ppm) 11.75 (brs, 1H), 7.36 (s, 1H), 7.07-7.09 (m, 1H), 7.00(d, 1H, J=8 Hz), 2.30 (s, 3H); ¹³C NMR (DMSO-d₆, 125 MHz): δ (ppm) 169.8, 133.9, 131.7, 127.0, 123.2, 122.5, 111.1, 20.5; MS (EI): m/z calcd for C₈H₇NOS [m]⁺: 165.0, found 165.0. m.p: 170-171° C.

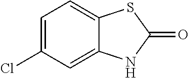 [SMILES: O=c1[nH]c2cc(Cl)ccc2s1]

Separated by dry packing and dry sample loading column chromatography (200-300 mesh silica gel): dichloromethane:ethyl acetate (V/V)=50:1 as developing agent, 94 mg of white solid was obtained with a yield after separation of 51.0%.

Characterization data of 5-chlorobenzothiazol-2-one: ¹H NMR (DMSO-d₆, 500 MHz): δ (ppm) 12.04 (brs, 1H), 7.61 (d, 1H, J=8.5 Hz), 7.19 (dd, 1H, J₁=8.5, J₂=2.5 Hz), 7.12 (d, 1H, J=2.0 Hz); ¹³C NMR (DMSO-d₆, 125 MHz): δ (ppm) 170.0, 137.4, 130.8, 124.3, 122.4, 122.2, 111.2; MS (EI): m/z calcd for C₇H₄ClNOS [M]⁺: 184.9, found 185.0. m.p.: 224-226° C.

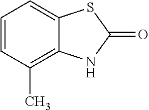

Separated by dry packing and dry sample loading column chromatography (200-300 mesh silica gel): dichloromethane:ethyl acetate (V/V)=5:1 as developing agent, 164 mg of white solid was obtained with a yield after separation of 99.3%.

Characterization data of 4-methylbenzothiazole-2-one: ¹H NMR (DMSO-d₆, 500 MHz): δ (ppm) 11.73 (brs, 1H), 7.37 (dd, 1H, J₁=7.5, J₂=0.5 Hz), 7.08-7.09 (m, 1H), 7.03 (t, 1H, J=7.5 Hz), 2.32 (s, 3H); ¹³C NMR (DMSO-d₆, 125 MHz): δ (ppm) 170.4, 135.0, 127.6, 122.8, 122.5, 121.3, 120.0, 17.4; MS (EI): m/z calcd for C₈H₇NOS [M]⁺: 165.1, found 165.0. m.p.: 211-212° C.

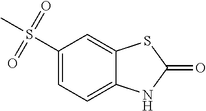

Separated by dry packing and dry sample loading column chromatography (200-300 mesh silica gel): ethyl acetate:petroleum ether (V/V)=3:1 as developing agent, 155 mg of white solid was obtained with a yield after separation of 67.9%.

Characterization data of methylsulfonyl benzothiazole-2-one: ¹H NMR(DMSO-d₆, 500 MHz): δ (ppm) 12.41 (brs, 1H), 8.22 (d, 1H, J=7.0 Hz), 7.812 (dd, 1H, J=7.5 Hz, J=2.0 Hz), 7.31 (d, 1H, J=8.0 Hz), 3.20 (s, 3H); ¹³C NMR (DMSO-d₆, 125 MHz): δ (ppm) 170.2, 140.4, 134.6, 125.6, 124.2, 122.2, 111.5, 43.9; MS (EI): m/z calcd for C₈H₇NO₃S₂ [M]⁺: 229.0, found 228.8. m.p.: 241-244° C.

### Example 6 Synthesis of Benzothiazolone Derivatives by the Reaction of Aromatic o-Aminodisulfides with CO₂ in the Presence of H₂S

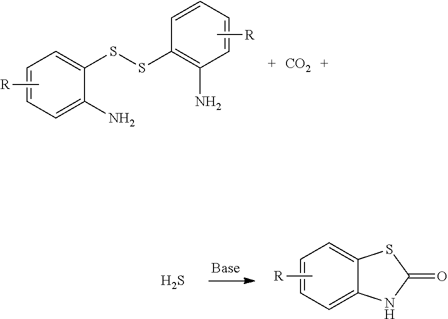

0.5 mmol of disulfide, 0.5 mmol of base, and 2 ml of solvent were added into the reactor sequentially, and the reactor was tightened, the appropriate amount of H₂S was introduced, preheated, and corresponding amount of CO₂ was introduced, then reacted at the corresponding temperature for 12 hours. After the reaction was completed, the reactor was cooled to room temperature, the gas in the reactor was slowly exhausted, then extracted with ethyl acetate and saturated salt water, the organic phases were combined and separated by column chromatography to obtain the target product.

The conditions were optimized according to the above steps, and the results were shown in the following table:

| | | | | CO₂ | H₂S | Tem- | |
|---|---|---|---|---|---|---|---|
| | | Molar ratio | | pres- | pres- | pera- | |
| | | (Sub- | Sol- | sure | sure | ture | Yield |
| Entry | base | strate:base) | vent | (MPa) | (MPa) | (° C.) | (%) |
| 1 | DBU | 1:1 | NMP | 3 | 0 | 50 | 0 |
| 2 | DBU | 1:1 | NMP | 3 | 0.2 | 50 | 87 |
| 3 | DBU | 1:1 | NMP | 3 | 0.3 | 50 | 97 |
| 4 | DBU | 1:1 | NMP | 3 | 0.4 | 50 | 97 |
| 5 | DBU | 1:1 | NMP | 3 | 0.6 | 50 | 94 |
| 6 | DBU | 1:1 | NMP | 3 | 0.8 | 50 | 96 |
| 7 | DBU | 1:1 | NMP | 3 | 1.0 | 50 | 94 |
| 8 | DBU | 1:0 | NMP | 3 | 0.3 | 50 | 0 |
| 9 | DBU | 1:0.4 | NMP | 3 | 0.3 | 50 | 92 |
| 11 | DBU | 1:2 | NMP | 3 | 0.3 | 50 | 84 |
| 12 | DBU | 1:1 | NMP | 1 | 0.3 | 50 | 83 |
| 13 | DBU | 1:1 | NMP | 0 | 0.3 | 50 | 0 |
| 14 | DBU | 1:1 | NMP | 5 | 0.3 | 50 | 92 |
| 15 | DBU | 1:1 | NMP | 3 | 0.3 | 25 | 90 |
| 16 | DBU | 1:1 | NMP | 3 | 0.3 | 80 | 86 |
| 17 | DBU | 1:1 | NMP | 3 | 0.3 | 100 | 97 |
| 18 | Et₃N | 1:1 | NMP | 3 | 0.3 | 50 | 75 |
| 19 | TMG | 1:1 | NMP | 3 | 0.3 | 50 | 90 |
| 20 | DBU | 1:1 | CH₃OH | 3 | 0.3 | 50 | 6.8 |
| 21 | DBU | 1:1 | 1,4- | 3 | 0.3 | 50 | 61 |
| | | | dioxane | | | | |
| 22 | DBU | 1:1 | DMSO | 3 | 0.3 | 50 | 34 |

Note:
the raw material was 0.5 mmol disulfide (dimer of o-aminothiophenol); the solvent was 2 mL; the molar ratio in the table was the mol ratio of disulfide:DBU; and the reaction time was 12 h.

### Using the Method as in Entry 3 while Changing Other Reaction Substrates, the Following Products were Obtained

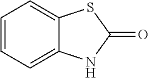

Separated by dry packing and dry sample loading column chromatography (200-300 mesh silica gel): dichloromethane:ethyl acetate (V/V)=20:1 as developing agent, 148 mg of white solid was obtained with a yield after separation of 98%.

Characterization data: ¹H NMR (CDCl₃, 500 MHz): δ (ppm) 10.01 (brs, 1H), 7.41 (d, 1H, J=7.5 Hz), 7.30-7.26 (m, 1H), 7.17-7.14 (m, 2H). ¹³C NMR (CDCl₃, 125 MHz): δ (ppm) 172.8, 135.3, 126.5, 123.9, 123.3, 122.6, 111.7; MS (EI): m/z calcd for C₇H₅NOS [M]⁺: 151.0, found 151.0. m.p.: 139-140° C.

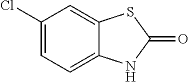 [SMILES: O=c1[nH]c2ccc(Cl)cc2s1]

Separated by dry packing and dry sample loading column chromatography (200-300 mesh silica gel): dichloromethane:ethyl acetate (V/V)=20:1 as developing agent, 180 mg of white solid was obtained with a yield after separation of 97.2%.

Characterization data: ¹H NMR (DMSO-d₆, 500 MHz): δ (ppm) 12.02 (brs, 1H), 7.74 (d, 1H, J=2.0 Hz), 7.32 (dd, 1H, J₁=8.5, J₂=2.5 Hz), 7.11 (d, 1H, J=8.5 Hz); ¹³C NMR (DMSO-d₆, 125 MHz): δ (ppm) 169.7, 135.3, 126.4, 125.2, 122.4, 122.7; MS (EI): m/z calcd for C₇H₄ClNOS [m]⁺: 185.1, found 185.0. m.p.: 212-214° C.

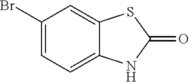 [SMILES: O=c1[nH]c2ccc(Br)cc2s1]

Separated by dry packing and dry sample loading column chromatography (200-300 mesh silica gel): dichloromethane:ethyl acetate (V/V)=20:1 as developing agent, 179 mg of white solid was obtained with a yield after separation of 78.3%.

Characterization data: ¹H NMR (DMSO-d₆, 500 MHz): δ (ppm) 12.02 (brs, 1H), 7.86 (d, 1H, J=2.0 Hz), 7.44 (dd, 1H, J₁=8.5, J₂=2.5 Hz), 7.05 (d, 1H, J=8.5 Hz); ¹³C NMR (DMSO-d₆, 125 MHz): δ (ppm) 169.7, 135.6, 129.2, 125.6, 125.0, 114.0, 113.1; MS (EI): m/z calcd for C₇H₄BrNOS [m]⁺: 228.9, found 228.9. m.p.: 231-232° C.

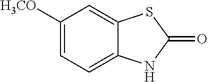 [SMILES: COc1ccc2[nH]c(=O)sc2c1]

Separated by dry packing and dry sample loading column chromatography (200-300 mesh silica gel): dichloromethane:ethyl acetate (V/V)=20:1 as developing agent, 169 mg of white solid was obtained with a yield after separation of 93.5%.

Characterization data: ¹H NMR (DMSO-d₆, 500 MHz): δ (ppm) 11.658 (brs, 1 H), 7.23 (d, 1H, J=2.5 Hz), 7.02 (d, 1H, J=8.5 Hz), 6.86 (dd, 1H, J₁=8.5 Hz, J₂=2.5 Hz), 3.73(s, 3H); ¹³C NMR (DMSO-d₆, 125 MHz): δ (ppm) 169.8, 155.2, 129.9, 124.3, 113.2, 112.1, 107.8, 55.6; MS (EI): m/z calcd for C₈H₇NO₂S [M]⁺: 180.9, found 181.0. m.p.: 161-163° C.

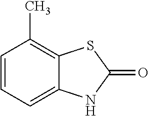

Separated by dry packing and dry sample loading column chromatography (200-300 mesh silica gel): dichloromethane:ethyl acetate (V/V)=20:1 as developing agent, 149 mg of white solid was obtained with a yield after separation of 90%.

Characterization data: ¹H NMR (DMSO-d₆, 500 MHz): δ (ppm) 11.73 (brs, 1H), 7.37 (dd, 1H, J₁=7.5 Hz, J₂=0.5 Hz), 7.08-7.09 (m, 1H), 7.03 (t, 1H, J=7.5 Hz), 2.32(s, 3H). ¹³C NMR (DMSO-d₆, 125 MHz): δ (ppm) 170.4, 135.0, 127.6, 122.8, 122.5, 121.3, 120.0, 17.4; MS (EI): m/z calcd for C₇H₅NOS [m]⁺: 165.1, found 165.0. m.p: 211-212° C.

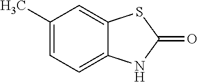

Separated by dry packing and dry sample loading column chromatography (200-300 mesh silica gel): dichloromethane:ethyl acetate (V/V)=20:1 as developing agent, 157 mg of white solid was obtained with a yield after separation of 95.4%.

Characterization data: ¹H NMR (DMSO-d₆, 500 MHz): δ (ppm) 11.75 (brs, 1H), 7.36 (s, 1H), 7.07-7.09 (m, 1H), 7.00(d, 1H, J=8 Hz), 2.30 (s, 3H); ¹³C NMR (DMSO-d₆, 125 MHz): δ (ppm) 169.8, 133.9, 131.7, 127.0, 123.2, 122.5, 111.1, 20.5; MS (EI): m/z calcd for C₈H₇NOS [m]⁺: 165.0, found 165.0. m.p.: 170-171° C.

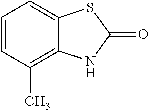

Separated by dry packing and dry sample loading column chromatography (200-300 mesh silica gel): dichloromethane:ethyl acetate (V/V)=20:1 as developing agent, 154 mg of white solid was obtained with a yield after separation of 93.5%.

Characterization data: ¹H NMR (DMSO-d₆, 500 MHz): δ (ppm) 11.73 (brs, 1H), 7.37 (dd, 1H, J₁=7.5, J₂=0.5 Hz), 7.08-7.09 (m, 1H), 7.03 (t, 1H, J=7.5 Hz), 2.32 (s, 3H); ¹³C NMR (DMSO-d₆, 125 MHz): δ (ppm) 170.4, 135.0, 127.6, 122.8, 122.5, 121.3, 120.0, 17.4; MS (EI): m/z calcd for c₈h₇NOS [m]⁺: 165.1, found 165.0. m.p: 211-212° C.

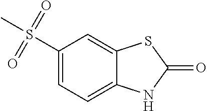

Separated by dry packing and dry sample loading column chromatography (200-300 mesh silica gel): dichloromethane:ethyl acetate (V/V)=1:2 as developing agent, 140 mg of white solid was obtained with a yield after separation of 61%.

Characterization data: ¹H NMR(DMSO-d₆, 500 MHz): δ (ppm) 12.41 (brs, 1H), 8.22 (d, 1H, J=7.0 Hz), 7.812 (dd, 1H, J=7.5 Hz, J=2.0 Hz), 7.31 (d, 1H, J=8.0 Hz), 3.20 (s, 3H); ¹³C NMR (DMSO-d₆, 125 MHz): δ (ppm) 170.2, 140.4, 134.6, 125.6, 124.2, 122.2, 111.5, 43.9; MS (EI): m/z calcd for C₈H₇NO₃S₂ [m]⁺: 229.0, found 228.8. m.p.: 241-244° C.

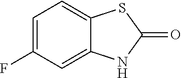 [SMILES: O=c1[nH]c2cc(F)ccc2s1]

Separated by dry packing and dry sample loading column chromatography (200-300 mesh silica gel): dichloromethane:ethyl acetate (V/V)=20:1 as developing agent, 128 mg of white solid was obtained with a yield after separation of 76%.

Characterization data: ¹H NMR (DMSO-d₆, 500 MHz): δ (ppm) 12.39 (brs, 1H), 7.43 (d, 1H, J=7.5 Hz), 7.12-7.22 (m, 2H). ¹³C NMR (DMSO-d₆, 125 MHz): δ (ppm) 169.7, 147.1(d, J=243.8 Hz), 125.5(d, J=3.8 Hz), 124.3(d, J=14.6 Hz), 123.0(d, J=6.5 Hz), 118.6(d, J=6.0 Hz), 112.7(d, J=16.9 Hz); MS (EI): M/z calcd for C₇H₅NOS [m]⁺: 169.0, found 169.0. m.p.: 172-174° C.

### Example 7 Synthesis of Imidazolidinone (Oxazolidinone or Thiazolidinone) Derivatives by the Reaction of Diamine, Alcoholamine or Mercaptoamine with CO₂ in the Presence of H₂S

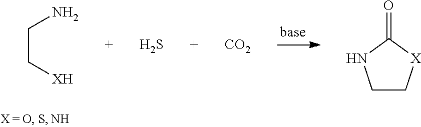

2 mmol of diamine, 0.8 mmol of base, and 2 mL of solvent were weighed and added to the reactor sequentially, and the reactor was tightened. The corresponding amount of H₂S was introduced into the reactor, and the corresponding amount of CO₂ was introduced at a suitable temperature, and then the mixture was stirred for 4 h. After the reaction was completed, the reactor was cooled to room temperature, and the reactor was opened after the gas in the reactor was slowly exhausted, then extracted, separated by column chromatography and recrystallized to obtain the target product.

The conditions were optimized according to the above steps, and the results were shown in the following table:

| | Base | | P(MPa) | | | | Yield |
|---|---|---|---|---|---|---|---|
| Entry | (equivalent) | Solvent | H₂S | CO₂ | Time(h) | T(° C.) | (%) |
| 1 | DBU(0.4) | NMP | 0.2 | 3 | 8 | 100 | 99 |
| 2 | DBU(0.4) | NMP | 0.2 | 3 | 4 | 100 | 99 |
| 3 | DBU(0.4) | NMP | 0.2 | 3 | 2 | 100 | 89 |
| 4 | DBU(0.4) | NMP | 0.2 | 3 | 1 | 100 | 80 |
| 5 | DBU(0.4) | NMP | 0.2 | 3 | 4 | 120 | 99 |
| 6 | DBU(0.4) | NMP | 0.2 | 1 | 4 | 110 | 93 |
| 7 | DBU(0.4) | NMP | 0.2 | 1 | 4 | 90 | 99 |
| 8 | DBU(0.4) | NMP | 0.2 | 1 | 4 | 90 | 88 |
| 9 | DBU(0.4) | NMP | 0.2 | 3 | 4 | 90 | 99 |
| 10 | DBU(0.4) | NMP | 0.2 | 5 | 4 | 90 | 89 |
| 11 | DBU(0.6) | NMP | 0.2 | 3 | 4 | 90 | 99 |
| 12 | DBU(0.4) | NMP | 0.2 | 3 | 4 | 90 | 99 |
| 13 | DBU(0.2) | NMP | 0.2 | 3 | 4 | 90 | 82 |
| 14 | DBU(0.4) | NMP | 0.4 | 3 | 4 | 90 | 96.4 |
| 15 | DBU(0.4) | NMP | 1 | 3 | 4 | 90 | 89.9 |
| 16 | DBU(0.4) | DMF | 0.2 | 3 | 4 | 90 | 98 |
| 17 | — | NMP | 0.2 | 3 | 12 | 90 | 79% |
| 18 | Et₃N(0.4) | NMP | 0.2 | 3 | 4 | 90 | 73% |
| 19 | DIPEA | NMP | 0.2 | 3 | 4 | 90 | 76% |
| 20 | NaSH (0.4) | NMP | 0.2 | 3 | 4 | 90 | 12% |
| 21 | K₂CO₃(0.4) | NMP | 0.2 | 3 | 4 | 90 | 8% |

Note:
the raw material was 2 mmol ethylenediamine; the solvent was 2 ml NMP. In entry 17, ethylenediamine was used as base and NMP was added as solvent.

### Using the Method as in Entry 10 While Changing Other Reaction Substrates, the Reaction Results are as Follows

### Characterization of Compounds

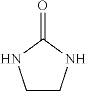

Extracted with ethyl acetate, separated by column chromatography and recrystallized, 170.2 mg of the pure target product was obtained with a yield of 99%.

Imidazolidin-2-one: white solid, ¹H NMR (500 MHz, CDCl₃) δ 3.52 (s, 1H). ¹³C NMR (126 MHz, CDCl₃) δ 165.64, 41.04.

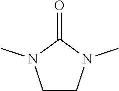

Extracted with ethyl acetate, separated by column chromatography and recrystallized, 153.7 mg of the pure target product was obtained with a yield of 68%. 1,3-Dimethylimidazolidin-2-one: Colorless oil, ¹HNMR (500 MHz, CDCl₃): d=2.79 (s,6H, 2CH₃), 3.27 (s, 4H,2CH₂); ¹³C NMR (126 MHz, CDCl₃): d=31.3, 44.9, 161.9.

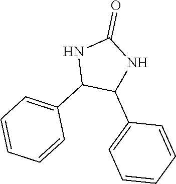 [SMILES: O=C1NC(c2ccccc2)C(c2ccccc2)N1]

Extracted with ethyl acetate, and separated by column chromatography, 216.5 mg of the pure target product was obtained with a yield of 91%.

4,5-Diphenylimidazolidin-2-one: white solid, ¹H NMR (500 MHz, CDCl₃): δ 7.38-7.34 (m, 6H), 7.27-7.30 (m, 4H), 5.83 (s, 2H), 4.57 (s, 2H). ¹³C NMR (126 MHz, CDCl₃) δ 163.1, 140.2, 128.7, 128.2, 126.4, 65.9;

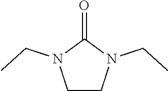

Extracted with ethyl acetate, and separated by multiple column chromatography, 241.74 mg of the pure target product was obtained with a yield of 85%.

1,3-Diethylimidazolidin-2-one: Colorless oil, 95%. ¹H NMR (500 MHz, CDCl₃): δ 3.23 (s, 4H), 3.19 (q, J=7.2 Hz, 4H), 1.05 (t, J=7.2 Hz, 6H). ¹³C NMR (500 MHz, CDCl₃): δ 161.3, 42.3, 38.9, 12.9.

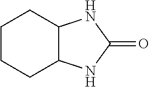

Extracted with ethyl acetate, and separated by column chromatography, 278.49 mg of the pure target product was obtained with a yield of 99%.

octahydro-2H-benzo[d]imidazol-2-one: colourless solid, ¹H NMR (500 MHz, CDCl₃) δ 4.75 (s, 2H), 3.67 (s, 2H), 1.66 (s, 4H), 1.61-1.49 (m, 2H), 1.31 (dt, J=9.6, 5.5 Hz, 2H); ¹³C NMR (126 MHz, CDCl₃) δ 77.67, 52.45, 28.85, 20.88.

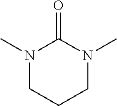

Extracted, and separated by column chromatography, 192 mg of the pure target product was obtained with a yield of 75%.

1,3-Dimethyl-3,4,5,6-tetrahydropyrimidin-2(1H)-one: ¹H NMR (500 MHz, CDCl₃): d=1.97 (quintet, J=6.0 Hz, 2H, CH₂), 2.92 (s, 6H, 2 CH₃), 3.24 (t, J=6.0 Hz, 4H, 2 CH₂); ¹³C NMR (126 MHz, CDCl₃): d=22.1, 35.5, 47.8, 156.7.

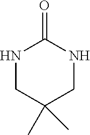

Filtrated, 188 mg of pure target product was obtained with a yield of 73%.

5,5-dimethyltetrahydropyrimidin-2(1H)-one: white solid, 1H NMR (500 MHz, DMSO-d6) δ 6.06 (s, 2H), 2.76 (s, 4H), 0.94 (s, 6H). ¹³C NMR (126 MHz, DMSO-d6) δ 155.26, 51.06, 27.20, 23.89.

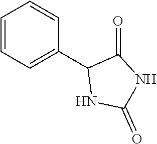 [SMILES: O=C1NC(=O)C(c2ccccc2)N1]

Extracted with ethyl acetate, and separated by column chromatography, 174.4 mg of the pure target product was obtained with a yield of 99%.

5-phenylimidazolidine-2,4-dione: white solid, 1H NMR (500 MHz, DMSO-d6) δ 10.77 (s, 1H), 8.39 (s, 1H), 7.37 (d, J=34.7 Hz, 6H), 5.16 (s, 1H). 13C NMR (126 MHz, DMSO-d6) δ 174.34, 157.65, 136.21, 128.80, 128.39, 126.86, 61.35.

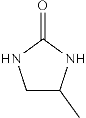

Spreated by column chromatography, and recrystallized with dichloromethane and ethyl acetate, 182 mg of the target product was obtained with a yield of 91%.

4-methylimidazolidin-2-one: white solid, 1H NMR (500 MHz, CDCl₃) δ 5.00 (s, 2H), 3.92 (h, J=8.3 Hz, 1H), 3.61 (t, J=8.4 Hz, 1H), 3.17-2.99 (m, 1H), 1.25 (d, J=6.4 Hz, 3H). ¹³C NMR (126 MHz, CDCl₃) δ 163.90, 48.41 , 48.01 , 21.15.

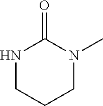

Filtrated, 164.1 mg of pure target product was obtained with a yield of 72%.

1-methyltetrahydropyrimidin-2(1H)-one: white solid, ¹H NMR (500 MHz, DMSO-d₆) δ 6.11 (s, 1H), 3.15 (t, J=5.5 Hz, 2H), 3.09 (t, J=5.5 Hz, 2H), 2.74 (s, 3H), 1.79 (p, J=6.6, 5.9 Hz, 2H). ¹³C NMR (126 MHz, DMSO-d₆) δ 46.95, 22.02

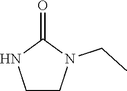

Extracted with ethyl acetate, and separated by column chromatography, 221.1 mg of the pure target product was obtained with a yield of 97%.

ethylimidazolidin-2-one: Colorless oil, ¹H NMR (500 MHz, CDCl₃) δ 3.50-3.35 (m, 6H), 3.25 (q, J=7.2 Hz, 2H), 1.12 (t, J=7.2 Hz, 3H). ¹³C NMR (126 MHz, CDCl₃) δ 162.59, 44.39, 38.25, 38.09, 12.69.

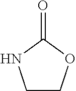

Extracted with ethyl acetate, and separated by column chromatography, 172.4 mg of the pure target product was obtained with a yield of 99%.

2-Oxazolidinone: 1H NMR (500 MHz, CDCl₃) δ 3.64 (t, 2H), 4.46 (t, 2H), 6.68 (s, 1H). ¹³CNMR (126 MHz, CDCl₃), δ 41.0, 65.5, 161.5.

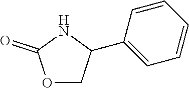 [SMILES: O=C1NC(c2ccccc2)CO1]

Separated by wet packing and dry sample loading column chromatography (200-300 mesh silica gel): methylene chloride and ethyl acetate (V/V=3:1) as developing agent, 280.6 mg of light yellow solid was obtained with a yield after separation of 86%.

4-phenyloxazolidin-2-one: ¹H NMR (500 MHz, CDCl₃) δ (ppm) 7.42-7.33 (m, 5H), 5.88 (s, 1H), 4.99-4.92 (m, 1H), 4.74 (t, J=8.7 Hz, 1H), 4.19 (dd, J=8.6, 7.0 Hz, 1H). ¹³C NMR (126 MHz, DMSO-d6) δ (ppm) 159.37, 141.48, 129.19, 128.44, 126.51, 71.84, 55.57. ESI-MS calcd for C₉H₁₀NO₂ [M+H]⁺ 164.06, found 164.10.

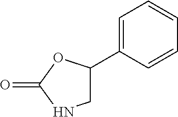 [SMILES: O=C1NCC(c2ccccc2)O1]

Separated by wet packing and dry sample loading column chromatography (200-300 mesh silica gel): methylene chloride and ethyl acetate (V/V=3:1) as developing agent, 229.5 mg of pale yellow solid was obtained with a yield after separation of 70%.

5-phenyloxazolidin-2-one: white solid, ¹H NMR (500 MHz, CDCl₃) δ (ppm) 7.43-7.36 (m, 5H), 5.77 (brs, 1H), 5.63 (t, J=8.1 Hz, 1H), 3.99 (t, J=9.0 Hz, 1H), 3.55 (t, J=8.4 Hz, 1H). ¹³C NMR (126 MHz, CDCl₃) δ (ppm) 159.66, 138.38, 128.92, 125.66, 77.90, 48.29. ESI-MS calcd for C₉H₁₀NO₂ [M+H]⁺ 164.06, found 164.10.

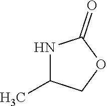

Separated by wet packing and dry sample loading column chromatography (200-300 mesh silica gel): methylene chloride and ethyl acetate (V/V=1:1) as developing agent, 201.6 mg of product was obtained with a yield after separation of 99%.

4-methyloxazolidin-2-one: ¹H NMR (500 MHz, CDCl₃) δ (ppm) 6.45 (brs, 1H), 4.50 (t, J=8.1 Hz, 1H), 4.05-3.99 (m, 1H), 3.96-3.93 (m, 1H), 1.30 (d, J=6.1 Hz, 3H). ¹³C NMR (126 MHz, CDCl₃) δ (ppm) 160.11, 71.65, 48.25, 20.78. ESI-MS calcd for C₄H₈NO₂ [M+H]⁺ 102.05, found 102.10.

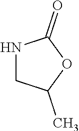

Separated by wet packing and dry sample loading column chromatography (200-300 mesh silica gel): methylene chloride and ethyl acetate (V/V=1:1) as developing agent, 182.2 mg of product was obtained with a yield after separation of 90%.

5-methyloxazolidin-2-one: ¹H NMR (500 MHz, CDCl₃) δ (ppm) 5.82 (brs, 1H), 4.81-4.75 (m, 1H), 3.71 (t, J=8.3 Hz, 1H), 3.21 (t, J=7.0 Hz, 1H), 1.46 (d, J=6.3 Hz, 3H). ¹³C NMR (126 MHz, CDCl₃) δ (ppm) 160.04, 77.21, 73.50, 47.40, 20.52. ESI-MS calcd for C₄H₈NO₂ [M+H]⁺ 102.05, found 102.10.

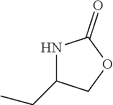

Separated by wet packing and dry sample loading column chromatography (200-300 mesh silica gel): methylene chloride and ethyl acetate (V/V=5:1) as developing agent, 225.6 mg of product was obtained with a yield after separation of 99%.

4-ethyloxazolidin-2-one: ¹H NMR (500 MHz, CDCl₃) δ (ppm) 5.80 (brs, 1H), 4.04 (dd, J=8.6, 6.0 Hz, 1H), 3.84-3.79 (m, 1H), 1.57-1.66 (m, 2H), 0.95 (t, J=7.5 Hz, 3H). ¹³C NMR (126 MHz, CDCl₃) δ (ppm) 159.73, 69.95, 53.75, 28.15, 9.29. ESI-MS calcd for C₅H₁₀NO₂ [M+H]⁺ 116.06, found 116.10.

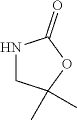

Separated by dry packing and dry sample loading column chromatography (200-300 mesh silica gel): methanol and methylene chloride (V/V=1:5) as developing agent, 225.9 mg of white solid was obtained with a yield after separation of 100%.

5,5-dimethyloxazolidin-2-one: ¹H NMR (500 MHz, CDCl₃) δ (ppm) 5.11(brs, 1H), 3.35 (s, 2H), 1.48 (s, 6H). ¹³C NMR (126 MHz, CDCl₃) δ (ppm) 159.34, 81.02, 52.64, 27.19. ESI-MS calcd for C₅H₁₀NO₂ [M+H]⁺ 116.06, found 116.15.

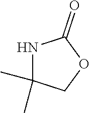

Separated by dry packing and dry sample loading column chromatography (200-300 mesh silica gel): methanol and methylene chloride (V/V=1:5) as developing agent, 93.5 mg of white solid was obtained with a yield after separation of 41%.

4,4-dimethyloxazolidin-2-one: ¹H NMR (500 MHz, CDCl₃) δ (ppm) 6.11 (brs, 1H), 4.09 (s, 2H), 1.37 (s, 6H). ¹³C NMR (126 MHz, CDCl₃) δ (ppm) 158.84, 76.90, 55.19, 27.59. ESI-MS calcd for C₅H₁₀NO₂ [M+H]⁺ 116.06, found 116.10.

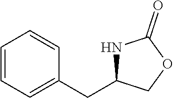 [SMILES: O=C1N[C@H](Cc2ccccc2)CO1]

Separated by wet packing and dry sample loading column chromatography (200-300 mesh silica gel): methylene chloride and ethyl acetate (V/V=2:1) as developing agent, 317.3 mg of white solid was obtained with a yield after separation of 90%.

(R)-4-benzyloxazolidin-2-one: ¹H NMR (500 MHz, DMSO-d6) δ (ppm) 7.78 (brs, 1H), 7.33-7.21 (m, 5H), 4.25 (t, J=8.3 Hz, 1H), 4.08-3.96 (m, 2H), 2.84-2.72 (m, 2H). ¹³C NMR (126 MHz, DMSO-d6) δ (ppm) 159.04, 136.99, 129.82, 128.84, 126.98, 68.46, 52.93, 40.68. ESI-MS calcd for C₁₀H₁₂NO₂ [M+H]⁺ 178.08, found 178.05.

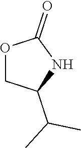

Separated by wet packing and wet sample loading column chromatography (200-300 mesh silica gel): methylene chloride and ethyl acetate (V/V=2:1) as developing agent, 237.2 mg of white solid was obtained with a yield after separation of 94%.

(S)-4-isopropyloxazolidin-2-one: ¹H NMR (500 MHz, CDCl₃) δ (ppm) 6.47(brs), 4.44 (t, J=8.7 Hz, 1H), 4.10 (dd, J=8.7, 6.3 Hz, 1H), 3.63-3.59 (m, 1H), 1.77-1.70 (m, 1H), 0.97 (d, J=6.7 Hz, 3H), 0.90 (d, J=6.8 Hz, 3H). ¹³C NMR (126 MHz, CDCl₃) δ (ppm) 160.25, 68.59, 58.34, 32.67, 17.99, 17.62. ESI-MS calcd for C₆H₁₂NO₂ [M+H]⁺ 130.08, found 130.10.

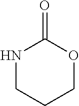

Extracted with ethyl acetate, and separated by column chromatography, 172.0 mg of the pure target product was obtained with a yield of 86%.

1,3-Oxazinan-2-one: ¹H NMR (500 MHz, DMSO d₆) δ 1.77-1.85 (m, 2H, NH—CH2-CH2-CH2-O), 3.12-3.19 (m, 2H), 4.15 (t, J=5.4 Hz, 2H), 7.13 (s, 1H,). ¹³C NMR (126 MHz, DMSO d₆) δ 21.79, 39.78, 67.04, 153.74.

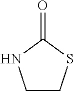

Extracted with ethyl acetate, and separated by column chromatography, 171.0 mg of the pure target product was obtained with a yield of 84%.

thiazolidin-2-one: ¹H NMR (500 MHz, CDCl₃) δ 3.37 (t, J=3.6 Hz, 2H), 3.59 (t, J=3.6 Hz, 2H), 6.99 (s, 1H)

### Example 8 Synthesis of Thioquinazolindione Derivatives by Reaction of o-Aminobenzonitrile and CO₂ in the Presence of H₂S

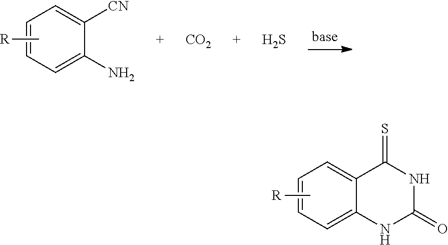

A magnet was placed into a 10 mL stainless steel high pressure reactor, and 1 mmol of o-aminobenzonitrile derivative, appropriate amount of H₂S and 2 ml of solvent were added sequentially, and the reactor was tightened. Carbon dioxide was introduced to the reactor at the specified pressure, stirred for 24h, then the reaction was ended and cooled down. The gas in the reactor was slowly exhausted, the reactor was opened and extracted with ethyl acetate and saturated salt water. The organic phases were combined and the crude product was obtained by distillation under reduced pressure. The pure target product was obtained by column chromatography (eluted with petroleum ether and ethyl acetate).

The conditions were optimized according to the above steps, and the reaction results are shown in the following table:

| | | | CO₂ | H₂S | | |
|---|---|---|---|---|---|---|
| | | | pres- | pres- | Tempera- | |
| | Molar ratio | | sure | sure | ture | Yield |
| Entry | (Substrate:base) | Solvent | (MPa) | (MPa) | (° C.) | (%) |
| 1 | 1:1 | DMF | 4 | 1 | 40 | 34 |
| 2 | 1:1 | DMF | 4 | 0.8 | 40 | 37 |
| 3 | 1:1 | DMF | 4 | 0.6 | 40 | 20 |
| 4 | 1:1 | DMF | 4 | 0.6 | 50 | 92 |
| 5 | 1:1 | DMF | 4 | 0.4 | 50 | 66 |
| 6 | 1:1 | DMF | 4 | 0.8 | 50 | 81 |
| 7 | 1:1 | DMF | 0 | 0.6 | 50 | 0 |
| 8 | 1:1 | DMF | 2 | 0.6 | 50 | 83 |
| 9 | 1:1 | DMF | 5 | 0.6 | 50 | 99 |
| 10 | 1:0.5 | DMF | 5 | 0.6 | 50 | 58 |
| 11 | 1:1.8 | DMF | 5 | 0.6 | 50 | 99 |
| 12 | 1:1 | NMP | 5 | 0.6 | 50 | 95 |

Note:
In each of the above reactions, the raw material was 1 mmol o-aminobenzonitrile; the solvent was 2 ml; the molar ratio was the molar ratio of raw material to DBU; and reacted for 24 h.

### Using the Method as in Entry 9 while Changing Other Reaction Substrates, the Following Products were Obtained

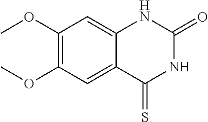 [SMILES: COc1cc2[nH]c(=O)[nH]c(=S)c2cc1OC]

Separated by dry packing and dry sample loading column chromatography (200-300 mesh silica gel) with gradient elution, petroleum ether and ethyl acetate as eluent, petroleum ether:ethyl acetate (V/V)=1:1, 235.2 mg of yellow solid 6,7-dimethoxy-2-oxo-4-thioquinazolindione was obtained with a yield after separation of 99%. The analysis results show that the obtained target product has a correct structure.

¹H NMR (500 MHz, DMSO-d₆) δ=12.50 (s, 1H), 11.46 (s, 1H), 7.69 (s, 1H), 6.64 (s, 1H), 3.84 (s, 3H), 3.79 (s, 3H). ¹³C NMR (126 MHz, DMSO-d₆) δ=188.97, 156.06, 147.50, 144.71, 134.94, 113.90, 110.09, 97.30, 56.05, 55.63. MS (ESI): m/z calcd for C₁₀H₁₀N₂O₃S [M]⁺: 239.04, found 239.2

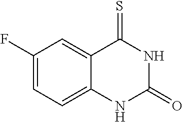 [SMILES: O=c1[nH]c(=S)c2cc(F)ccc2[nH]1]

Separated by dry packing and dry sample loading column chromatography (200-300 mesh silica gel) with gradient elution, petroleum ether and ethyl acetate as eluent, petroleum ether:ethyl acetate (V/V)=2:1, and the polarity is increased to 1:1, 184 mg of yellow solid 6-fluoro-2-oxo-4-thioquinazolindione was obtained with a yield after separation of 94%. The analysis results shown that the obtained target product has a correct structure.

¹H NMR (500 MHz, DMSO-d₆) δ=12.91 (s, 1H), 11.67 (s, 1H), 7.96 (dd, J=9.7, 3.0 Hz, 1H), 7.58 (td, J=8.5, 3.0 Hz, 1H), 7.20 (dd, J=9.0, 4.6 Hz, 1H). ¹³C NMR (126 MHz, DMSO-d₆) δ=190.86 (d, J=3.3 Hz), 157.63 (d, J=240.0 Hz), 147.13, 134.92, 123.63 (d, J=24.9 Hz), 120.94 (d, J=8.19 Hz), 118.26 (d, J=8.06 Hz), 114.76 (d, J=25.3 Hz). MS (ESI): m/z calcd for C₈H₅FN₂OS [M]⁺: 197.01, found 196.9

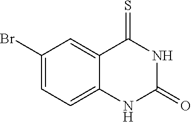 [SMILES: O=c1[nH]c(=S)c2cc(Br)ccc2[nH]1]

Separated by dry packing and dry sample loading column chromatography (200-300 mesh silica gel) with gradient elution, petroleum ether and ethyl acetate as eluent, petroleum ether:ethyl acetate (V/V)=3:1, 217 mg of white solid 6-bromo-2-oxo-4-thioquinazolindione was obtained with a yield after separation of 85%, and the analysis results shown that the obtained target product has a correct structure.

¹H NMR (500 MHz, DMSO-d₆) δ=12.94 (s, 1H), 11.75 (s, 1H), 8.37 (dd, J=2.4, 1.0 Hz, 1H), 7.83 (ddd, J=8.6, 2.4, 1.0 Hz, 1H), 7.13 (dd, J=8.7, 1.1 Hz, 1H). ¹³C NMR (126 MHz, DMSO-d₆) δ=189.50, 147.08, 138.29, 137.44, 132.05, 121.64, 118.36, 114.79. MS (ESI): m/z calcd for C₈H₅BrN₂OS [M]⁺: 257.9, found 257.1.

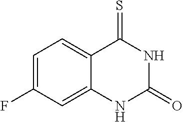 [SMILES: O=c1[nH]c(=S)c2ccc(F)cc2[nH]1]

Separated by dry packing and dry sample loading column chromatography (200-300 mesh silica gel) with gradient elution, petroleum ether and ethyl acetate as eluent, petroleum ether:ethyl acetate (V/V)=2:1, 162 mg of yellow solid 7-fluoro-2-oxo-4-thioquinazolindione was obtained with a yield after separation of 83%. The analysis results show that the obtained target product has a correct structure.

¹H NMR (500 MHz, DMSO-d₆) δ=12.77 (s, 1H), 11.65 (s, 1H), 8.27 (t, J=7.3 Hz, 1H), 7.59 (dd, J=8.8, 6.6 Hz, 1H), 7.37-7.29 (m, 1H). ¹³C NMR (126 MHz, DMSO-d₆) δ=190.80, 162.40, 150.31, 146.01, 141.27, 127.28, 120.12, 111.07 (d, J=11.3 Hz). MS (ESI): m/z calcd for C₈H₅FN₂OS [M]⁺:, 197.01 found 197.3.

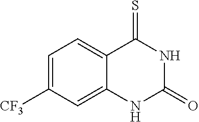 [SMILES: O=c1[nH]c(=S)c2ccc(C(F)(F)F)cc2[nH]1]

Separated by dry packing and dry sample loading column chromatography (200-300 mesh silica gel) with gradient elution, petroleum ether and ethyl acetate as eluent, petroleum ether:ethyl acetate (V/V)=2:1, 219mg of yellow solid 7-trifluoromethyl-2-oxo-4-thioquinazolindione was obtained with a yield after separation of 89%, and the analysis results shown that the obtained target product has a correct structure.

¹H NMR (500 MHz, DMSO-d₆) δ=13.05 (s, 1H), 11.81 (s, 1H), 8.47 (d, J=8.5 Hz, 1H), 7.53-7.47 (m, 1H), 7.45 (d, J=1.7 Hz, 1H). ¹³C NMR (126 MHz, DMSO-d₆) δ=191.30, 147.06, 138.48, 134.17 (q, J=32.8 Hz), 132.04, 123.34 (q, J=273.7 Hz), 122.36, 118.84 (q, J=3.8 Hz), 113.15 (q, J=3.8 Hz). MS (ESI): m/z calcd for C₉H₅F₃N₂OS [M]⁺: 247.01, found 247.3.

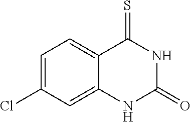 [SMILES: O=c1[nH]c(=S)c2ccc(Cl)cc2[nH]1]

Separated by dry packing and dry sample loading column chromatography (200-300 mesh silica gel) with gradient elution, petroleum ether and ethyl acetate as eluent, petroleum ether:ethyl acetate (V/V)=2:1, 138 mg of yellow solid 7-chloro-2-oxo-4-thioquinazolindione was obtained with a yield after separation of 65%, and the analysis results shown that the obtained target product has a correct structure.

¹H NMR (500 MHz, DMSO-d₆) δ=12.71 (s, 1H), 11.56 (s, 1H), 8.23 (t, J=8.4 Hz, 1H), 7.48 (dd, J=8.5, 1.2 Hz, 1H), 6.54-6.40 (m, 1H). ¹³C NMR (126 MHz, DMSO-d₆) δ=191.92, 170.17, 151.67, 136.49, 130.76, 115.32, 113.52, 112.55. MS (ESI): m/z calcd for C₈H₅ClN₂OS [M]⁺: 212.65, found 212.3.

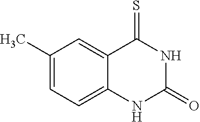

Separated by dry packing and dry sample loading column chromatography (200-300 mesh silica gel) with gradient elution, petroleum ether and ethyl acetate as eluent, petroleum ether:ethyl acetate (V/V)=2:1, 172 mg of yellow solid 7-methyl-2-oxo-4-thioquinazolindione was obtained with a yield after separation of 89%, and the analysis results shown that the obtained target product has a correct structure.

¹H NMR (500 MHz, DMSO-d₆) δ=12.66 (s, 1H), 11.56 (s, 1H), 8.19 (d, J=8.3 Hz, 1H), 7.03 (d, J=8.4 Hz, 1H), 6.94 (s, 1H), 2.35 (s, 3H). ¹³C NMR (126 MHz, DMSO-d₆)) δ=188.97, 148.23, 146.65, 137.75, 131.11, 125.24, 119.85, 114.69. MS (ESI): m/z calcd for C₉H₈N₂OS [M]⁺: 193.04, found 193.2.

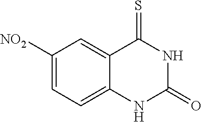 [SMILES: O=c1[nH]c(=S)c2cc([N+](=O)[O-])ccc2[nH]1]

Separated by dry packing and dry sample loading column chromatography (200-300 mesh silica gel) with gradient elution, petroleum ether and ethyl acetate as eluent, petroleum ether:ethyl acetate (V/V)=1:1, 80 mg of brown solid 6-nitro-2-oxo-4-thioquinazolindione was obtained with a yield after separation of 36%, and the analysis results shown that the obtained target product has a correct structure.

¹H NMR (500 MHz, DMSO-d₆) δ=12.51 (s, 1H), 11.28 (s, 1H), 7.48 (d, J=2.6 Hz, 1H), 7.00 (dd, J=8.7, 2.6 Hz, 1H), 6.92-6.89 (m, 1H). ¹³C NMR (126 MHz, DMSO-d₆) δ=191.01, 158.80, 144.50, 128.55, 123.74, 122.13, 116.09, 111.20. MS (ESI): m/z calcd for C₈H₅N₃O₃S [M]⁺: 223.01, found 223.4.

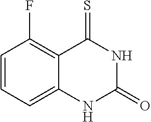 [SMILES: O=c1[nH]c(=S)c2c(F)cccc2[nH]1]

Separated by dry packing and dry sample loading column chromatography (200-300 mesh silica gel) with gradient elution, petroleum ether and ethyl acetate as eluent, petroleum ether:ethyl acetate (V/V)=2:1, 133 mg of yellow solid 5-fluoro-2-oxo-4-thioquinazolindione was obtained with a yield after separation of 68%, and the analysis results shown that the obtained target product has a correct structure.

¹H NMR (500 MHz, DMSO-d₆) δ=11.85 (s, 1H), 11.20 (s, 1H), 7.35 (t, J=8.1 Hz, 1H), 6.68 (d, J=8.3 Hz, 1H), 6.50 (d, J=7.8 Hz, 1H). ¹³C NMR (126 MHz, DMSO-d₆) δ=186.47, 154.09, 147.11, 141.12, 134.24, 111.58, 110.66, 104.63. MS (ESI): m/z calcd for C₈H₅FN₂OS [M]⁺: 197.01, found 197.2

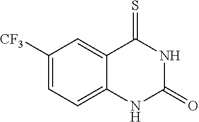 [SMILES: O=c1[nH]c(=S)c2cc(C(F)(F)F)ccc2[nH]1]

Separated by dry packing and dry sample loading column chromatography (200-300 mesh silica gel) with gradient elution, petroleum ether and ethyl acetate as eluent, petroleum ether:ethyl acetate (V/V)=2:1, 240 mg of yellow solid 6-trifluoromethyl-2-oxo-4-thioquinazolindione was obtained with a yield after separation of 97%, and the analysis results shown that the obtained target product has a correct structure.

¹H NMR (500 MHz, DMSO-d₆) δ=13.06 (s, 1H), 11.97 (d, J=3.8 Hz, 1H), 8.57-8.53 (m, 1H), 8.01-7.94 (m, 1H), 7.34 (dd, J=8.6, 3.8 Hz, 1H). ¹³C NMR (126 MHz, DMSO-d₆) δ=191.24, 147.13, 141.10, 131.40 (q, J=3.0 Hz), 127.52 (q, J=4.4 Hz), 123.84 (q, J=272.2 Hz), 123.36 (q, J=32.8 Hz), 119.82, 117.43. MS (ESI): m/z calcd for C₉H₅F₃N₂OS [M]⁺: 247.01, found 247.3.

### Example 10 Synthesis of Substituted Urea Derivatives by Reaction of Benzylamine and CO₂ in the Presence of H₂S

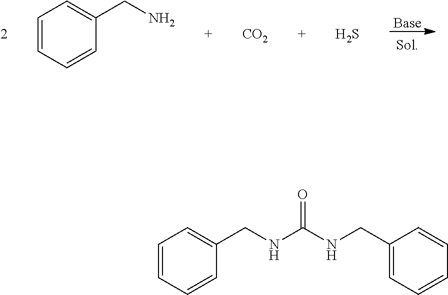

2 mmol of benzylamine, DBU and 1 mL of suitable solvent were added sequentially in a 15 mL high-pressure reactor, and the reactor was tightened; the required amount of H₂S and CO₂ gas was sequentially introduced into the reactor; finally, the reactor was continued to be stirred at a suitable temperature for 24 hours; after the reaction was completed, a certain amount of distilled water was added to the reaction mixture to precipitate the product completely, and then filtered and dried sequentially to obtain the product.

The conditions were optimized according to the above steps, and the reaction results were shown in the following table:

| | Base | | n(mmol) | | | | Yield |
|---|---|---|---|---|---|---|---|
| Entry | (equivalent) | T(° C.) | H₂S | CO₂ | Solvent | Time(h) | (%) |
| 1 | DBU(0.5) | 100 | 1 | 10 | NMP | 24 | 64 |
| 2 | DBU(0) | 100 | 1 | 10 | NMP | 24 | 75 |
| 3 | DBU(0) | 110 | 1 | 10 | NMP | 24 | 85 |
| 4 | DBU(0) | 120 | 1 | 10 | NMP | 24 | 91 |
| 5 | DBU(0) | 130 | 1 | 10 | NMP | 24 | 86 |
| 6 | DBU(0) | 120 | 2 | 10 | NMP | 24 | 84 |
| 7 | DBU(0) | 120 | 0.5 | 10 | NMP | 24 | 72 |
| 8 | DBU(0) | 120 | 0 | 10 | NMP | 24 | NR |
| 9 | DBU(0) | 120 | 1 | 6 | NMP | 24 | 71 |
| 10 | DBU(0) | 120 | 1 | 20 | NMP | 24 | 80 |
| 11 | DBU(0) | 120 | 1 | 10 | NMP | 36 | 71 |
| 12 | DBU(0) | 120 | 1 | 10 | NMP | 12 | 59 |

Note:
In each of the above reactions, the raw material was 2 mmol benzylamine; the solvent was 1 ml, NR: no reaction.

### Using the Method as in Entry 4 while Changing Other Reaction Substrates, the Following Products were Obtained

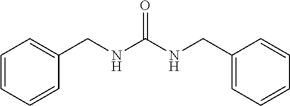 [SMILES: O=C(NCc1ccccc1)NCc1ccccc1]

205 mg of white powder product was obtained by filtration and drying, with a yield of 91%.

¹H NMR (500 MHz, DMSO-d₆) δ 7.31 (t, J=7.5 Hz, 4H), 7.28-7.18 (m, 6H), 6.43 (t, J=6.1 Hz, 2H), 4.23 (d, J=6.0 Hz, 4H).

¹³C NMR (126 MHz, DMSO-d₆) δ 158.08, 140.89, 128.19, 126.96, 126.52, 42.98.

MS (ESI): m/z calcd for C₁₅H₁₇N₂O [M+H]⁺: 241.10, found 241.13, m.p.: 168-169° C.

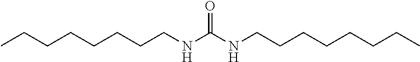 [SMILES: CCCCCCCCNC(=O)NCCCCCCCC]

244 mg of light yellow powder product was obtained by filtration and drying, with a yield of 86%.

¹H NMR (500 MHz, Chloroform-d) δ 4.58 (s, 2H), 3.18-3.10 (m, 4H), 1.47 (q, J=6.8 Hz, 4H), 1.28 (d, J=10.4 Hz, 20H), 0.88 (t, J=6.4 Hz, 6H).

¹³C NMR (126 MHz, Chloroform-d) δ 158.50, 40.63, 31.83, 30.31, 29.36, 29.27, 26.96, 22.66, 14.09.

MS (ESI): m/z calcd for C₁₉H₄₀N₃O [M+H+CH₃CN]⁺: 326.30, found 326.32, m.p.: 89-91° C.

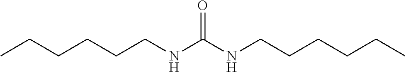 [SMILES: CCCCCCNC(=O)NCCCCCC]

163 mg of white powder product was obtained by filtration and drying, with a yield of 71%.

¹H NMR (500 MHz, DMSO-d₆) δ 5.72 (t, J=5.7 Hz, 2H), 2.94 (q, J=6.8, 6.4 Hz, 4H), 1.32 (q, J=6.8 Hz, 4H), 1.27-1.18 (m, 12H), 0.85 (t, J=6.7 Hz, 6H).

¹³C NMR (126 MHz, Chloroform-d) δ 158.62, 40.58, 31.59, 30.29, 26.63, 22.60, 14.03.

MS (ESI): m/z calcd for C₁₃H₂₉N₂O [M+H]⁺: 229.20, found 229.23, m.p.: 73-76° C.

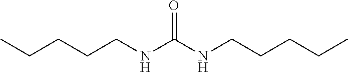

129 mg of white powder product was obtained by filtration and drying, with a yield of 64%.

¹H NMR (500 MHz, DMSO-d₆) δ 5.71 (t, J=5.9 Hz, 2H), 2.95 (q, J=6.7 Hz, 4H), 1.34 (p, J=7.1 Hz, 4H), 1.31-1.18 (m, 8H), 0.86 (t, J=7.1 Hz, 6H).

¹³C NMR (126 MHz, DMSO-d₆) δ 158.52, 40.55, 30.19, 29.06, 22.35, 14.39.

MS (ESI): m/z calcd for C₁₁H₂₅N₂O [M+H]⁺: 201.15, found 201.20, m.p.: 86-88° C.

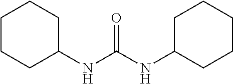 [SMILES: O=C(NC1CCCCC1)NC1CCCCC1]

After 36 h of reaction, 157 mg of white crystalline product was obtained by filtration and drying, with a yield of 73%.

¹H NMR (500 MHz, TFA-d) δ 5.11 (s, 2H), 3.54 (d, J=9.8 Hz, 4H), 3.35 (d, J=10.0 Hz, 4H), 3.22 (d, J=11.6 Hz, 2H), 2.86 (ddt, J=42.0, 22.0, 11.2 Hz, 12H).

¹³C NMR (126 MHz, TFA-d) δ 159.93, 55.32, 35.04, 27.30, 26.93.

MS (ESI): m/z calcd for C₁₃H₂₅N₂O [M+H]⁺: 225.10, found 225.20, m.p.: 229-230° C.

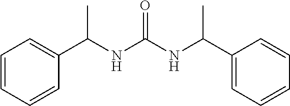 [SMILES: CC(NC(=O)NC(C)c1ccccc1)c1ccccc1]

243 mg of light yellow powder product was obtained by filtration and drying, with a yield of 91%.

¹H NMR (500 MHz, DMSO-d₆) δ 7.35-7.15 (m, 10H), 6.27 (d, J=8.1 Hz, 2H), 4.72 (q, J=7.0 Hz, 2H), 1.30 (dd, J=10.8, 7.5 Hz, 6H).

¹³C NMR (126 MHz, DMSO-d₆) δ 156.54, 145.67, 128.27, 126.50, 125.72, 48.50, 23.40.

MS (ESI): m/z calcd for C₁₇H₂₁N₂O [M+H]⁺: 269.10, found 269.17, m.p.: 122-123° C.

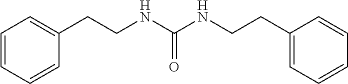 [SMILES: O=C(NCCc1ccccc1)NCCc1ccccc1]

244 mg of silvery white powder product was obtained by filtration and drying, with a yield of 91%.

¹H NMR (500 MHz, DMSO-d₆) δ 7.29 (t, J=7.5 Hz, 4H), 7.23-7.16 (m, 6H), 5.89 (t, J=5.1 Hz, 2H), 3.22 (q, J=6.7 Hz, 4H), 2.67 (d, J=7.2 Hz, 4H).

¹³C NMR (126 MHz, DMSO-d₆) δ 157.97, 139.81, 128.72, 128.35, 126.02, 40.97, 36.23.

MS (ESI): m/z calcd for C₁₇H₂₁N₂O [M+H]⁺: 269.10, found 269.17, m.p.: 138-140° C.

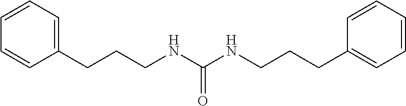 [SMILES: O=C(NCCCc1ccccc1)NCCCc1ccccc1]

285 mg of white powder product was obtained by filtration and drying, with a yield of 96%.

¹H NMR (500 MHz, DMSO-d₆) δ 7.26 (t, J=7.5 Hz, 2H), 7.17 (dd, J=15.1, 7.4 Hz, 3H), 5.88 (d, J=5.3 Hz, 1H), 2.98 (t, J=6.6 Hz, 2H), 2.58-2.53 (m, 2H), 1.66 (q, J=7.3 Hz, 2H).

¹³C NMR (126 MHz, DMSO-d₆) δ 158.22, 141.91, 128.33, 128.32, 125.74, 38.84, 32.58, 31.93.

MS (ESI): m/z calcd for C₁₉H₂₅N₂O [M+H]⁺: 297.20, found 297.20, m.p.: 92-93° C.

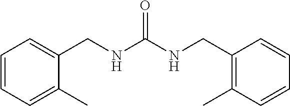 [SMILES: Cc1ccccc1CNC(=O)NCc1ccccc1C]

233 mg of white powder product was obtained by filtration and drying, with a yield of 86%.

¹H NMR (500 MHz, DMSO-d₆) δ 7.20 (d, J=4.7 Hz, 1H), 7.14 (s, 3H), 6.27 (t, J=5.5 Hz, 1H), 4.21 (d, J=4.9 Hz, 2H), 2.26 (s, 3H).

¹³C NMR (126 MHz, DMSO-d₆) δ 157.93, 138.37, 135.45, 129.94, 127.22, 126.73, 125.77, 41.02, 18.58.

MS (ESI): m/z calcd for C₁₇H₂₁N₂O [M+H]⁺: 269.10, found 269.17, m.p.: 237-238° C.

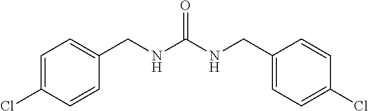 [SMILES: O=C(NCc1ccc(Cl)cc1)NCc1ccc(Cl)cc1]

260 mg of white crystalline product was obtained by filtration and drying, with a yield of 84%.

¹H NMR (500 MHz, DMSO-d₆) δ 7.35 (d, J=8.3 Hz, 4H), 7.26 (d, J=8.2 Hz, 4H), 6.66 (t, J=5.8 Hz, 2H), 4.20 (d, J=5.7 Hz, 4H).

¹³C NMR (126 MHz, DMSO-d₆) δ 158.20, 140.09, 131.09, 128.88, 128.19, 42.34.

MS (ESI): m/z calcd for C₁₅H₁₆Cl₂N₂NaO₂ [M+Na+H₂O]⁺: 350.05, found 350.05, m.p.: 253-254° C.

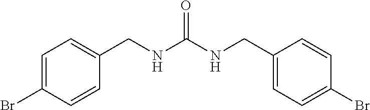 [SMILES: O=C(NCc1ccc(Br)cc1)NCc1ccc(Br)cc1]

344 mg a offwhite crystalline product was obtained by filtration and drying, with a yield of 86%.

¹H NMR (500 MHz, DMSO-d₆) δ 7.49 (d, J=7.9 Hz, 4H), 7.19 (d, J=7.9 Hz, 4H), 6.53 (t, J=5.8 Hz, 2H), 4.18 (d, J=6.0 Hz, 4H).

¹³C NMR (126 MHz, DMSO-d₆) δ 158.10, 140.50, 131.11, 129.27, 119.56, 42.42.

MS (ESI): m/z calcd for C₁₅H₁₆Br₂N₂NaO₂ [M+Na+H₂O]⁺: 439.95, found 439.95, m.p.: 268-270° C.

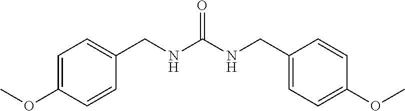

257 mg of white powder product was obtained by filtration and drying, with a yield of 85%

¹H NMR (500 MHz, DMSO-d₆) δ 7.17 (d, J=8.5 Hz, 4H), 6.86 (d, J=8.6 Hz, 4H), 6.30 (s, 2H), 4.14 (s, 4H), 3.72 (s, 6H).

¹³C NMR (126 MHz, DMSO-d₆) δ 158.15, 132.79, 128.40, 113.70, 55.13, 42.50.

MS (ESI): m/z calcd for C₁₇H₂₁N₂O₃ [M+H]⁺: 301.10, found 301.16, m.p.: 178-180° C.

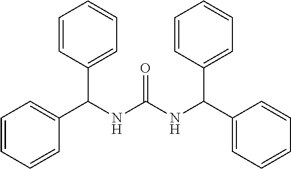 [SMILES: O=C(NC(c1ccccc1)c1ccccc1)NC(c1ccccc1)c1ccccc1]

290 mg of light yellow solid product was obtained by filtration and drying, with a yield of 74%.

¹H NMR (500 MHz, DMSO-d₆) δ 7.31 (d, J=6.9 Hz, 8H), 7.28-7.18 (m, 12H), 6.95 (d, J=8.1 Hz, 2H), 5.88 (d, J=8.0 Hz, 2H).

¹³C NMR (126 MHz, DMSO-d₆) δ 156.79, 144.01, 128.87, 127.25, 127.22, 57.41.

MS (ESI): m/z calcd for C₂₇H₂₅N₂O [M+H]⁺: 393.15, found 393.20, m.p.: 283-284° C.

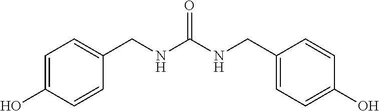 [SMILES: O=C(NCc1ccc(O)cc1)NCc1ccc(O)cc1]

106 mg of off-white powder product was obtained by filtration and drying, with a yield of 39%.

¹H NMR (500 MHz, DMSO-d₆) δ 9.23 (s, 2H), 7.04 (d, J=8.1 Hz, 4H), 6.69 (d, J=7.2 Hz, 4H), 6.18 (d, J=6.0 Hz, 2H), 4.09 (d, J=5.8 Hz, 4H).

¹³C NMR (126 MHz, DMSO-d₆) δ 157.97, 156.08, 130.92, 128.38, 114.95, 42.57.

MS (ESI): m/z calcd for C₁₅H₁₇N₂O₃ [M+H]⁺: 273.05, found 273.12, m.p.: 185-187° C.

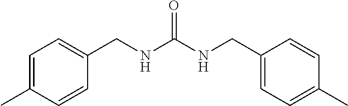

240 mg of white crystalline product was obtained by filtration and drying, with a yield of 89%.

¹H NMR (500 MHz, DMSO-d₆) δ 7.17-7.08 (m, 8H), 6.33 (t, J=6.1 Hz, 2H), 4.17 (d, J=6.0 Hz, 4H), 2.27 (s, 6H).

¹³C NMR (126 MHz, DMSO-d₆) δ 157.91, 137.68, 135.41, 128.62, 126.87, 42.59, 20.54.

MS (ESI): m/z calcd for C₁₇H₂₁N₂O [M+H]⁺: 269.10, found 269.17, m.p.: 216-218° C.

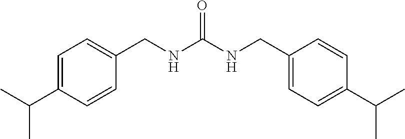 [SMILES: CC(C)c1ccc(CNC(=O)NCc2ccc(C(C)C)cc2)cc1]

203 mg of white solid product product was obtained by filtration and drying, with a yield of 63%.

¹H NMR (500 MHz, DMSO-d₆) δ 7.16 (s, 8H), 6.33 (t, J=6.0 Hz, 2H), 4.18 (d, J=5.9 Hz, 4H), 2.85 (hept, J=6.7 Hz, 2H), 1.18 (d, J=6.9 Hz, 12H).

¹³C NMR (126 MHz, DMSO-d₆) δ 157.92, 146.59, 138.11, 126.98, 125.97, 42.67, 33.01, 23.87.

MS (ESI): m/z calcd for C₂₁H₂₉N₂O [M+H]⁺: 325.15, found 325.23, m.p.: 122-123° C.

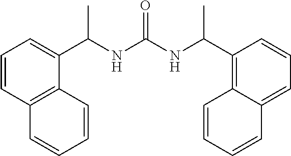 [SMILES: CC(NC(=O)NC(C)c1cccc2ccccc12)c1cccc2ccccc12]

287 mg of white powder product was obtained by filtration and drying, with a yield of 78%.

¹H NMR (500 MHz, DMSO-d₆) δ 8.15 (d, J=8.4 Hz, 1H), 8.08 (d, J=9.5 Hz, 1H), 7.97-7.89 (m, 2H), 7.85-7.77 (m, 2H), 7.60-7.42 (m, 8H), 6.42 (dd, J=20.3, 8.1 Hz, 2H), 5.55 (h, J=6.9 Hz, 2H), 1.46 (dd, J=22.3, 6.9 Hz, 6H).

¹³C NMR (126 MHz, DMSO-d₆) δ 156.36, 141.29, 133.41, 130.42, 128.57, 127.14, 126.04, 125.54, 123.24, 121.84, 44.65, 22.53.

MS (ESI): m/z calcd for C₂₅H₂₅N₂O [M+H]⁺: 369.15, found 369.20, m.p.: 223-225° C.

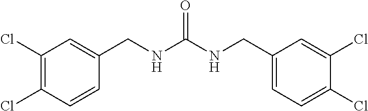 [SMILES: O=C(NCc1ccc(Cl)c(Cl)c1)NCc1ccc(Cl)c(Cl)c1]

33.1 mg of light yellow solid product was obtained by filtration and drying, with a yield of 87%.

¹H NMR (500 MHz, DMSO-d₆) δ 7.56 (d, J=8.2 Hz, 2H), 7.46 (d, J=2.0 Hz, 2H), 7.24 (dd, J=8.3, 2.0 Hz, 2H), 6.69 (t, J=6.2 Hz, 2H), 4.21 (d, J=6.1 Hz, 4H).

¹³C NMR (126 MHz, DMSO-d₆) δ 157.97, 142.39, 130.80, 130.36, 128.96, 128.83, 127.29, 41.96.

MS (ESI): m/z calcd for C₁₅H₁₄ClN₂NaO₂ [M+Na+H₂O]⁺: 419.95, found 419.97, m.p.: 174-176° C.

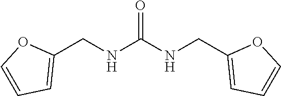 [SMILES: O=C(NCc1ccco1)NCc1ccco1]

196 mg of white powder product was obtained by filtration and drying, with a yield of 89%.

¹H NMR (500 MHz, DMSO-d₆) δ 7.55 (s, 2H), 6.40-6.30 (m, 4H), 6.18 (d, J=3.2 Hz, 2H), 4.20 (d, J=5.7 Hz, 4H).

¹³C NMR (126 MHz, DMSO-d₆) δ 157.40, 153.54, 141.89, 110.41, 106.22, 40.02, 39.85, 39.69, 39.52, 39.35, 39.19, 39.02, 36.37.

MS (ESI): m/z calcd for C₁₁H₁₃N₂O₃ [M+H]⁺: 221.00, found 221.09, m.p.: 126-128° C.

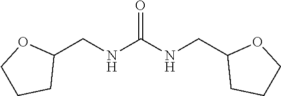 [SMILES: O=C(NCC1CCCO1)NCC1CCCO1]

A yellow oil was obtained by separation column with a yield of 89%.

¹H NMR (500 MHz, DMSO-d₆) δ 6.08-6.02 (m, 2H), 3.75 (dq, J=13.9, 6.4 Hz, 4H), 3.59 (q, J=7.5 Hz, 2H), 3.10 (dq, J=14.3, 5.2 Hz, 2H), 3.05-2.95 (m, 2H), 1.89-1.72 (m, 6H), 1.52 - 1.41 (m, 2H).

¹³C NMR (126 MHz, DMSO-d₆) δ 158.68, 78.10, 67.40, 43.56, 28.43, 25.50.

MS (ESI): m/z calcd for C₁₁H₂₁N₂O₃ [M+H]⁺: 229.10, found 229.16, Pyrolysis temperature: 140° C.

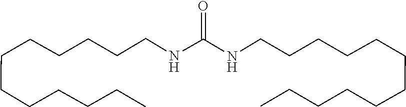 [SMILES: CCCCCCCCCCCCNC(=O)NCCCCCCCCCCCC]

370 mg of yellow solid product was obtained by filtration and drying, with a yield of 93%.

¹H NMR (500 MHz, TFA-d) δ 4.92 (t, J=7.1 Hz, 4H), 3.23 (p, J=6.9 Hz, 4H), 2.90 (d, J=31.6 Hz, 36H), 2.43 (td, J=6.7, 2.9 Hz, 6H).

¹³C NMR (126 MHz, TFA-d) δ 160.26, 109.99, 43.49, 33.13, 30.76, 30.65, 30.55, 30.51, 30.18, 29.82, 27.62, 23.70, 14.05.

MS (ESI): m/z calcd for C₂₅H₅₂KN₂O [M+K]⁺: 435.30, found 435.37, m.p.: 105-106° C.

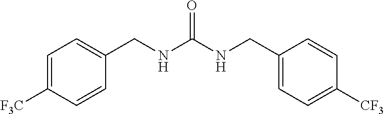 [SMILES: O=C(NCc1ccc(C(F)(F)F)cc1)NCc1ccc(C(F)(F)F)cc1]

311 mg of white solid product product was obtained by filtration and drying, with a yield of 83%.

¹H NMR (500 MHz, DMSO-d₆) δ 7.67 (d, J=8.0 Hz, 4H), 7.46 (d, J=7.9 Hz, 4H), 6.69 (t, J=6.2 Hz, 2H), 4.32 (d, J=6.0 Hz, 4H).

¹³C NMR (126 MHz, DMSO-d₆) δ 158.14, 145.99, 127.57, 127.30(q, J=32.1 Hz), 125.09(q, J=3.9 Hz), 124.44(q, J=272.4 Hz), 42.67.

MS (ESI): m/z calcd for C₁₇H₁₅F₆N₂O [M+H]⁺: 377.05, found 377.11, m.p.: 106-161° C.

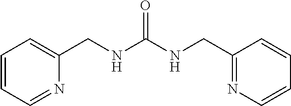 [SMILES: O=C(NCc1ccccn1)NCc1ccccn1]

A light yellow oil was obtained by separation column with a yield of 92%.

¹H NMR (500 MHz, DMSO-d₆) δ 8.50 (d, J=4.7 Hz, 2H), 7.76 (t, J=7.6 Hz, 2H), 7.30 (d, J=7.8 Hz, 2H), 7.28-7.21 (m, 2H), 6.75 (t, J=5.8 Hz, 2H), 4.34 (d, J=5.8 Hz, 4H).

¹³C NMR (126 MHz, DMSO-d₆) δ 159.69, 158.12, 148.69, 136.62, 121.90, 120.84, 44.96.

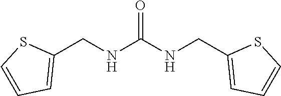 [SMILES: O=C(NCc1cccs1)NCc1cccs1]

197 mg of brown yellow powder product was obtained by filtration and drying, with a yield of 78%.

¹H NMR (500 MHz, DMSO-d₆) δ 7.36 (d, J=4.7 Hz, 2H), 6.93 (d, J=4.3 Hz, 4H), 6.50 (t, J=6.0 Hz, 2H), 4.38 (d, J=5.8 Hz, 4H).

¹³C NMR (126 MHz, DMSO-d₆) δ 157.40, 144.22, 126.60, 124.66, 38.12.

MS (ESI): m/z calcd for C₁₁H₁₃N₂OS₂ [M+H]⁺: 253.00, found 253.05, m.p.: 163-165° C.

All documents referred to in the present invention are incorporated by reference herein as if each document is individually incorporated by reference. Further, it should be understood that upon reading the above teaching of the present invention, various modifications or modifications may be made to the present invention by those skilled in the art, and those equivalents also fall within the scope defined by the appended claims of the present application.

1. A method for preparing carbonyl compounds using carbon dioxide as a carbonylation reagent, wherein the method is performed in the presence of H₂S and a optional base.

2. The method of claim 1, wherein the method comprises step (i) or step (ii): 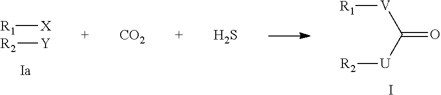
(i) in an optional inert solvent, reacting a compound of formula Ia with CO₂ in the presence of optional a base and an inorganic sulfur reagent to obtain a compound of formula I; 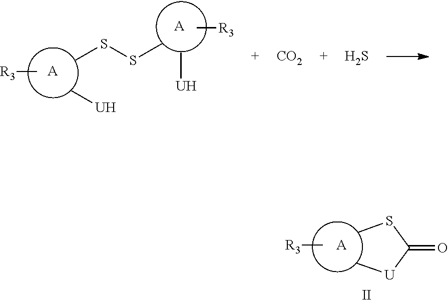
(ii) in an optional inert solvent, reacting a compound of formula IIa with CO₂ in the presence of a base and an inorganic sulfur reagent to obtain a compound of formula II;
Wherein, R₁ and R₂ are each independently selected from the group consisting of: substituted or unsubstituted C₁-C₁₂ alkyl (e.g. substituted or unsubstituted C₁-C₆ alkyl, substituted or unsubstituted C₁-C₈ alkyl), substituted or unsubstituted C₃-C₈ cycloalkyl, substituted or unsubstituted C₂-C₆ alkenyl, and substituted or unsubstituted C₂-C₆ alkynyl; or R₁ and R₂ together form a group selected from the group consisting of: substituted or unsubstituted C₁-C₆ alkylene, substituted or unsubstituted C₆-C₁₀ aryl, and substituted or unsubstituted 5-12-membered heteroaryl;
ring A is substituted or unsubstituted C₆-C₁₀aryl, or substituted or unsubstituted 5-12-membered heteroaryl;
X and Y are independently selected from the group consisting of: halogen, CN, SH, OH, NH₂, NHR, and NO₂;
U and V are independently selected from the group consisting of: NR, S, O, and —C(═S)NH;
R is selected from the group consisting of: H, substituted or unsubstituted C₁-C₁₂ alkyl (such as substituted or unsubstituted C₁-C₆ alkyl, substituted or unsubstituted C₁-C₈ alkyl), substituted or unsubstituted C₃-C₈ cycloalkyl, substituted or unsubstituted C₁-C₆ alkoxy, SO₂CH₃, and phenyl unsubstituted or substituted with 1-4 substituents selected from the group consisting of: halogen, C₁-C₆ alkyl, C₁-C₆ haloalkyl, C₁-C₆ alkoxy, OH, NO₂, NH₂, and SO₂CH₃.
R₃ is one or more groups on the ring A and selected from the group consisting of: H, halogen, C₁-C₆ alkyl, C₁-C₆ alkoxy, C₁-C₆ haloalkyl, NH₂, NO₂, SO₂CH₃, and phenyl unsubstituted or substituted with 1-4 substituents selected from the group consisting of: halogen, C₁-C₆ alkyl, C₁-C₆haloalkyl, C₁-C₆alkoxy, OH, NO₂, NH₂, SO₂CH₃; or R₅ and R₆ together form a —(CH₂)ₙ—, wherein, n is selected from 2, 3, 4, 5 or 6;
and the substituted means that one or more hydrogen atoms on the group are substituted with a substituent selected from the group consisting of: halogen, oxygen atom (i.e., ═O), C₁-C₆ alkyl, C₂-C₆ alkenyl, C₂-C₆ alkynyl, C₁-C₆ alkoxy, C₁-C₆ haloalkyl, NO₂, SO₂CH₃, phenyl, 5-12-membered heteroaryl, 3-8-membered cycloalkyl, 5-12-membered saturated or partially unsaturated heterocycle; wherein, the phenyl, heteroaryl, cycloalkyl or heterocycle is unsubstituted or substituted by 1-4 substituents selected from the group consisting of: halogen, C₁-C₆ alkyl, C₁-C₆ haloalkyl, C₁-C₆alkoxy, OH, NO₂, NH₂, and SO₂CH₃;
or, two substituents adjacent or attached to the same carbon atom together form a —(CH₂)ₙ—, wherein, n is selected from 2, 3, 4, 5 or 6.

3. The method of claim 1, wherein the base is an organic base; preferably, the base is selected from the group consisting of: C₁-C₁₂tertiary amines, C₁-C₁₂secondary amines, C₁-C₁₂primary amines, C₂-C₁₂ amidines, C₂-C₁₂ guanidines, C₃-C₁₂ pyridines, C₃-C₁₂ imidazoles; preferably, the base is selected from the group consisting of: DBU, TBD, MTBD, DBN, TMG, DABCO, ethylenediamine (EDA), triethylamine (EtN₃), diisopropylethylamine (DIPEA), DMAP, pyridine, and combinations thereof; preferably, the molar ratio of the reaction substrate to the base is 1:0-5 (e. g., 1:0.1-5).

4. The method of claim 1, wherein the method comprises steps (a), (b), (c), (d), (e), (f) or (g); 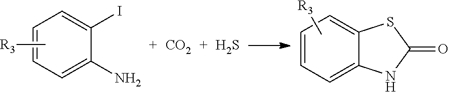
(a) in an optional inert solvent, reacting an o-iodoaniline with CO₂ and H₂S in the presence of a base to obtain a benzothiazolone derivative; 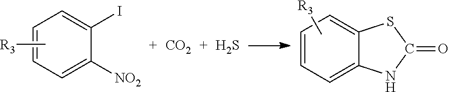
(b) in an optional inert solvent, reacting an o-nitroiodobenzene with CO₂ and H₂S in the presence of a base to synthesize a benzothiazolone derivative; 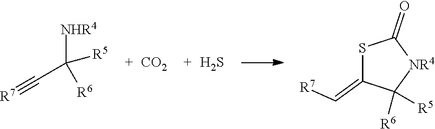
(c) in an optional inert solvent, reacting a propargylamine derivative with CO₂ and H₂S in the presence of an optional base to synthesize a thiazolidin-2-one derivative;
wherein, R₄ is selected from the group consisting of: H, substituted or unsubstituted C₁-C₁₂alkyl, substituted or unsubstituted C₃-C₈cycloalkyl, and substituted or unsubstituted phenyl;
R₅, R₆ and R₇ are independently selected from the group consisting of: H, substituted or unsubstituted C₁-C₁₂alkyl, substituted or unsubstituted C₃-C₈cycloalkyl, phenyl, 5-12 membered heteroaryl, and 5-12 membered saturated or partially unsaturated heterocycle, and the phenyl, heteroaryl or heterocycle is unsubstituted or substituted with 1-4 substituents selected from the group consisting of: halogen, C₁-C₆alkyl, C₁-C₆haloalkyl, C₁-C₆alkoxy, OH, NO₂, NH₂, SO₂CH₃; or R₅ and R₆ together form a —(CH₂)ₙ—, wherein, n is selected from 2, 3, 4, 5 or 6; 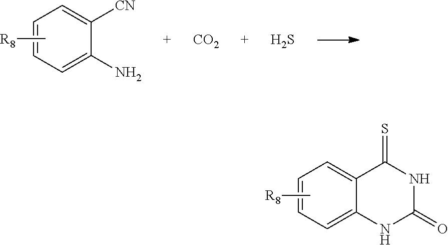
(d) in an optional inert solvent, reacting an o-aminobenzonitrile with CO₂ and H₂S in the presence of a base to synthesize a thioquinazolindione derivative;
wherein, R₈ is one or more substituents on the benzene ring and selected from the group consisting of: H, halogen, C₁-C₆alkyl, C₁-C₆alkoxy, C₁-C₆haloalkyl, NO₂, SO₂CH₃, and phenyl unsubstituted or substituted with 1-4 substituents selected from the group consisting of: halogen, C₁-C₆alkyl, C₁-C₆haloalkyl, C₁-C₆alkoxy, OH, NO₂, NH₂, and SO₂CH₃; 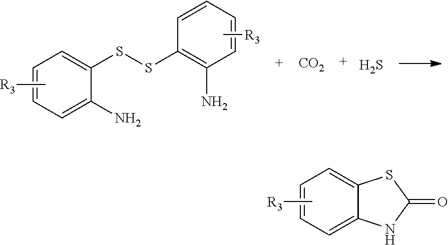
(e) in an optional inert solvent, in the presence of a base, reacting an aromatic o-aminodisulfide with CO₂ in the presence of H₂S to synthesize a benzothiazolone derivative; 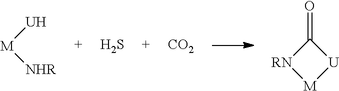
(f) In an optional inert solvent, in the presence of an optional base, reacting an diamine, an alcoholamine or a mercaptoamine with CO₂ in the presence of H₂S to synthesize an imidazolidinone derivative, an oxazolidinone derivative or a thiazolidinone derivative; wherein U is O, S or NR;
M is substituted or unsubstituted C₂-C₄alkylene, substituted or unsubstituted phenyl, or substituted or unsubstituted 5-12 membered heteroaryl, wherein the definition of the substituted is as described in claim 2;
(g) in an optional inert solvent, in the presence of an optional base, reacting an amine with CO₂ in the presence of H₂S to synthesize a urea derivative; 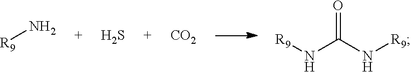
each R₉ are selected from the group consisting of: H, substituted or unsubstituted C₁-C₁₂alkyl (such as substituted or unsubstituted C₁-C₆alkyl, substituted or unsubstituted C₁-C₈alkyl), substituted or unsubstituted C₃-C₈cycloalkyl, phenyl, 5-12-membered heteroaryl, and 5-12-membered saturated or partially unsaturated heterocycle, and the phenyl, heteroaryl or heterocycle is unsubstituted or substituted with 1-4 substituents selected from the group consisting of: halogen, C₁-C₆alkyl, C₁-C₆haloalkyl, C₁-C₆alkoxy, OH, NO₂, NH₂, and SO₂CH₃.

5. The method of claim 1, wherein the inert solvent is selected from the group consisting of NMP, DMF, THF, DMSO, 1,4-dioxane, HMPA, CH₂Cl₂, CHCl₃, CCl₄, toluene, ethyl acetate, supercritical CO₂, and combinations thereof.

6. The method of claim 1, wherein in the reaction, the molar ratio of the reaction substrate to the CO₂ is 1:1-100.

7. The method of claim 1, wherein during the reaction, the CO₂ is continuously introduced into the reactor, and the pressure of the CO₂ in the reactor is 0.1-12 MPa.

8. The method of claim 1, wherein in the reaction, the molar ratio of the reaction substrate to H₂S is 1:0.05-20.

9. The method according to claim 1, wherein during the reaction, H₂S is continuously introduced into the reactor, and the pressure of H₂S in the reactor is 0.05-1.5 MPa.

10. The method according to claim 1, wherein the reaction temperature is from room temperature to 150° C.
